SEQUENCE LISTING (26 sequences)

SEQ ID NO: 1 - protein, 216 aa, Artificial Sequence
  synthetic sequence
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 2 - protein, 445 aa, Artificial Sequence
  synthetic sequence
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 14 aa, Artificial Sequence
  synthetic sequence
TGTGSDVGSYNLVS

SEQ ID NO: 4 - protein, 7 aa, Artificial Sequence
  synthetic sequence
GDSERPS

SEQ ID NO: 5 - protein, 10 aa, Artificial Sequence
  synthetic sequence
SSYAGSGIYV

SEQ ID NO: 6 - protein, 5 aa, Artificial Sequence
  synthetic sequence
TYAMG

SEQ ID NO: 7 - protein, 14 aa, Artificial Sequence
  synthetic sequence
SIGASGSQTRYADS

SEQ ID NO: 8 - protein, 7 aa, Artificial Sequence
  synthetic sequence
LAIGDSY

SEQ ID NO: 9 - protein, 110 aa, Artificial Sequence
  synthetic sequence
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVL

SEQ ID NO: 10 - protein, 116 aa, Artificial Sequence
  synthetic sequence
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSS

SEQ ID NO: 11 - skipped

SEQ ID NO: 12 - protein, 5 aa, Artificial Sequence
  synthetic sequence
NYAMG

SEQ ID NO: 13 - skipped

SEQ ID NO: 14 - protein, 14 aa, Artificial Sequence
  synthetic sequence
SIGASGAQTRYADS

SEQ ID NO: 15 - protein, 14 aa, Artificial Sequence
  synthetic sequence
SIGASGGQTRYADS

SEQ ID NO: 16-22 - skipped (7 entries)

SEQ ID NO: 23 - protein, 445 aa, Artificial Sequence
  synthetic sequence
EVQLLESGGGLVQPGGSLRLSCAASGFTFSTYAMGWVRQAPGKGLEWVSSIGSSGAQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 445 aa, Artificial Sequence
  synthetic sequence
EVQLLESGGGLVQPGGSLRLSCAASGFTFSDYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 445 aa, Artificial Sequence
  synthetic sequence
EVQLLESGGGLVQPGGSLRLSCAASGFTFSNYAMGWVRQAPGKGLEWVSSIGASGAQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 445 aa, Artificial Sequence
  synthetic sequence
EVQLLESGGGLVQPGGSLRLSCAASGFTFSTYAMGWVRQAPGKGLEWVSSIGASGGQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2024/0182598 A1
(43) Pub. Date: Jun. 6, 2024

(54) COMPOSITIONS AND METHODS FOR TREATING PEDIATRIC MYASTHENIA GRAVIS

(71) Applicant: Momenta Pharmaceuticals, Inc., Titusville, NJ (US)

(72) Inventors: Shawn Black, Devon, PA (US); Sindhu Ramchandren, Knoxville, TN (US); Yaowei Zhu, Chester Springs, PA (US)

(21) Appl. No.: 18/286,455

(22) PCT Filed: Apr. 12, 2022
(86) PCT No.: PCT/US2022/024354

§ 371 Date: Oct. 11, 2023

Related U.S. Application Data
(60) Provisional application No. 63/266,880, filed on Jan. 18, 2022.
(60) Provisional application No. 63/219,155, filed on Jul. 7, 2021.
(60) Provisional application No. 63/173,919, filed on Apr. 12, 2021.

(51) Int. Cl. C07K 16/42; A61K 39/00; A61K 47/02; A61K 47/26

(52) U.S. Cl. CPC C07K 16/4208; A61K 47/02; A61K 47/26; A61K 2039/505; A61K 2039/545; A61K 2039/55; C07K 2317/565; C07K 2317/76

(57) ABSTRACT

Composition and methods for treating pediatric myasthenia gravis are provided herein using compositions comprising anti-FcRn antibodies.

### CROSS-REFERENCE TO RELATED APPLICATIONS

This application claims priority to U.S. Provisional Application No. 63/173,919, filed Apr. 12, 2021, U.S. Provisional Application No. 63/219,155, filed Jul. 7, 2021, and U.S. Provisional Application No. 63/266,880, filed Jan. 18, 2022, each of which is hereby incorporated by reference in its entirety.

### SEQUENCE LISTING

The instant application contains a Sequence Listing which has been submitted electronically in ASCII format and is hereby incorporated by reference in its entirety. Said ASII copy, created on Apr. 7, 2022, is named 258199_001202_ST25.txt and is 26,081 bytes in size.

### FIELD

The present application is related to treating pediatric patients with myasthenia gravis.

### BACKGROUND

With a prevalence of 15 to 25 cases per 100,000 individuals and an annual incidence of 0.8 to 1 case per 100,000 individuals, myasthenia gravis (MG) is a rare, heterogeneous, neuromuscular disease characterized by fluctuating, fatigable muscle weakness. Weakness most often affects ocular, bulbar, proximal extremity, neck, and respiratory muscles, fluctuates during the day, and worsens with fatigue, repetitive activities, heat, infection, and stress. In most cases, initial symptoms are ocular and include ptosis and diplopia, but within 2 to 3 years of onset, the disease usually worsens and other muscles become affected; this is referred to as generalized MG (gMG). Additional symptoms typically include difficulty chewing, dysphagia, dysarthria, hypophonia, dyspnea, an inability to hold the mouth closed, a “snarling” expression when attempting to smile, an appearance of sadness or sleepiness, difficulty holding the head upright, and weakness in the hands and feet. Disease progression is associated with considerable morbidity due to aspiration, an increased incidence of respiratory infections and of falls, and side effects of immunosuppressant therapies. In addition, respiratory muscle weakness can lead to myasthenic crisis, which can be life threatening and require hospitalization, mechanical ventilation, tube feeding, fast acting immunosuppressive agents, and intensive care. Myasthenic crisis is also associated with additional infectious and cardiovascular complications, which contribute to morbidity and the risk of mortality. A significant unmet need exists for treatment options for patients suffering from MG. The embodiments provided for herein fulfil these needs as well as others.

### SUMMARY

In some embodiments, a method of treating pediatric myasthenia gravis in a pediatric patient in need thereof, is provided. In some embodiments, the method comprises administering an initial loading dose of about 30 mg/kg to about 60 mg/kg of an anti-FcRn antibody followed by administering a maintenance dose of about 15 mg/kg to about 30 mg/kg of the anti-FcRn antibody, wherein the anti-FcRn antibody comprises: a heavy chain comprising a HCDR1 of SEQ ID NO: 6, a HCDR2 of SEQ ID NO: 7, and a HCDR3 of SEQ ID NO: 8; and a light chain comprising a LCDR1 of SEQ ID NO: 3, a LCDR2 of SEQ ID NO: 4, and a LCDR3 of SEQ ID NO: 5; wherein the administration reduces serum IgG in the patient by at least 10%, at least 20%, at least 30%, at least 40%, at least 50%, at least 60%, at least 70%, at least 80%, or at least 90% of baseline serum IgG, and wherein the pediatric myasthenia gravis is selected from transient neonatal myasthenia, juvenile myasthenia gravis, congenital myasthenia gravis syndrome, or any combination thereof.

In some embodiments, a pharmaceutical composition comprising an anti-FcRn antibody for administration to a pediatric patient suffering from pediatric myasthenia gravis, is provided. In some embodiments, the pharmaceutical composition comprising the anti-FcRn antibody is administered to the pediatric patient intravenously or subcutaneously at an initial loading dose of about 30 mg/kg to about 60 mg/kg followed by administering a maintenance dose of about 15 mg/kg to about 30 mg/kg of the anti-FcRn antibody; and the anti-FcRn antibody comprises: a heavy chain comprising a HCDR1 of SEQ ID NO: 6, a HCDR2 of SEQ ID NO: 7, and a HCDR3 of SEQ ID NO: 8; and a light chain comprising a LCDR1 of SEQ ID NO: 3, a LCDR2 of SEQ ID NO: 4, and a LCDR3 of SEQ ID NO: 5, and wherein the pediatric myasthenia gravis is selected from transient neonatal myasthenia, juvenile myasthenia gravis, congenital myasthenia gravis syndromes, or any combination thereof.

### BRIEF DESCRIPTION OF THE DRAWINGS

FIG. 1 is a graph showing the results of modeling designed to predict the IgG reduction and MG-ADL improvement with 15 mg/kg and 30 mg/kg q2w maintenance dosing in adult patients with gMG.

FIG. 2 is a graph showing the results of modeling designed to predict the IgG reduction and MG-ADL improvement with a 30 mg/kg loading dose in adult patients with gMG.

### DETAILED DESCRIPTION

This application incorporates by reference each of the following in their entirety: PCT Application No. PCT/US2021/058188, filed Nov. 5, 2022, U.S. Provisional Application No. 63/110,884, filed Nov. 6, 2020, U.S. Provisional Application No. 63/137,972, filed Jan. 15, 2021, U.S. Provisional Application No. 63/173, 126, filed Apr. 9, 2021, U.S. Provisional Application No. 63/173,919, filed Apr. 12, 2021, U.S. Provisional Application No. 63/174,423, filed Apr. 13, 2021, U.S. Provisional Application No. 63/175,440, filed Apr. 15, 2021, U.S. Provisional Application No. 63/203,075, filed Jul. 7, 2021, U.S. Provisional Application No. 63/203,077, filed Jul. 7, 2021, U.S. Provisional Application No. 63/219,155, filed Jul. 7, 2021, and U.S. Provisional Application No. 63/266,880, filed Jan. 18, 2022.

Myasthenia gravis is caused by pathogenic autoantibodies that impair cholinergic transmission in the postsynaptic membrane at the neuromuscular junction and impair or prevent muscle contraction. In approximately 85% of cases, circulating antibodies target the acetylcholine receptor (AChR) itself. Up to half of the remaining 15% of patients have antibodies against muscle-specific tyrosine kinase (MuSK), an enzyme critical for neuromuscular junction formation and agrin induced AChR clustering, while approximately 7% to 8% of patients have neither anti-AChR nor anti-MuSK antibodies and have historically been considered “seronegative”. In this latter group, approximately 10% have pathogenic autoantibodies against lipoprotein-related protein receptor 4, an end plate protein that, along with MuSK, serves as an agrin receptor and is required for AChR clustering and normal neuromuscular junction formation.

The present disclosure provides for embodiments for methods of treating myasthenia gravis and related symptoms or pathologies associated with the same. In some embodiments, a pediatric subject with myasthenia gravis is treated with an antibody against human neonatal Fc receptor (FcRn). Anti-FcRn antibodies are useful, e.g., to promote clearance of autoantibodies in a pediatric subject, to suppress antigen presentation in a pediatric subject, to block an immune response, e.g., block an immune complex-based activation of the immune response in a pediatric subject, or to treat immunological diseases (e.g., autoimmune diseases) in a pediatric subject.

In some embodiments, the anti-FcRn antibody is nipocalimab, which can also be referred to as M281. In some embodiments, the term “nipocalimab” and the term “M281” are used interchangeably. Nipocalimab is an antibody to human neonatal Fc receptor (FcRn). Nipocalimab is described in U.S. Pat. No. 10,676,526, PCT Publication No. WO2020/023310, PCT Publication No. WO2020/018910, and PCT Publication No. WO2021/022249, each of which are hereby incorporated by reference in their entirety.

The term “antibody” herein is used in the broadest sense and encompasses various antibody structures, including but not limited to monoclonal antibodies, polyclonal antibodies, multispecific antibodies (e.g., bispecific antibodies), scFVs, nanobodies, VHH, and antibody fragments so long as they exhibit FcRn antigen-binding activity.

Additionally, antibody or antibody molecule, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs).

Immunoglobulin chains exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)). In some embodiments, the antibodies provided herein comprise the same FRs and different CDRs. In some embodiments, the antibodies provided herein comprise the same CDRs and different FRs. In some embodiments, mutations in the FR are in the heavy chain. In some embodiments, mutations in the FR are in the FR1 of the heavy chain. In some embodiments, mutations in the FR are in the FR2 of the heavy chain. In some embodiments, mutations in the FR are in the FR3 of the heavy chain. In some embodiments, mutations in the FR are in the FR4 of the heavy chain. In some embodiments, mutations in the FR are in the light chain. In some embodiments, mutations in the FR are in the FR1 of the light chain. In some embodiments, mutations in the FR are in the FR2 of the light chain. In some embodiments, mutations in the FR are in the FR3 of the light chain. In some embodiments, mutations in the FR are in the FR4 of the light chain. In some embodiments, mutations in the FR are in the heavy and light chains. In some embodiments, mutations in the FR are in any one or more of the FRs of the heavy and light chains.

The term “antibody molecule” also encompasses whole or antigen binding fragments of domain, or single domain, antibodies, which can also be referred to as “sdAb” or “VHH.” Domain antibodies comprise either V_{H} or V_{L} that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in PCT Publication No. WO2004041862 and PCT Publication No. WO2016065323, each of which is hereby incorporated by reference.

In some embodiments, as provided for herein, antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as a glycine/serine, glycine/alanine linker or other types of peptide linkers generally known to link the two domains together. Two peptide sequences are linked directly if they are directly connected to one another or indirectly if there is a linker or other structure that links the two regions. A linker can be directly linked to two different peptide sequences or domains.

As used herein, the terms “variable region” and “variable domain” refer to the portions of the light and heavy chains of an antibody that include amino acid sequences of complementary determining regions (CDRs, e.g., CDR L1, CDR L2, CDR L3, CDR H1, CDR H2, and CDR H3) and framework regions (FRs). According to the methods used in this disclosure, the amino acid positions assigned to CDRs and FRs are defined according to Kabat (Sequences of Proteins of Immunological Interest, 5th Ed. Public Health Service, National Institutes of Health, Bethesda, MD. (1991)). Using this numbering system, the actual linear amino acid sequence may contain fewer or additional amino acids corresponding to a shortening of, or insertion into, a CDR (defined further herein) or FR (defined further herein) of the variable region. For example, a heavy chain variable region may include a single inserted residue (i.e., residue 52a according to Kabat) after residue 52 of CDR H2 and inserted residues (i.e., residues 82a, 82b, 82c, etc. according to Kabat) after residue 82 of heavy chain FR. The Kabat numbering of residues may be determined for a given antibody by alignment at regions of homology of the sequence of the antibody with a “standard” Kabat numbered sequence.

As used herein, the terms “complementary determining regions” and “CDRs” refer to the regions of an antibody variable domain or variable region which are hypervariable in sequence and/or form structurally defined loops. A CDR is also known as a hypervariable region. The light chain and heavy chain variable regions each has three CDRs. The light chain variable region contains CDR L1, CDR L2, and CDR L3. The heavy chain variable region contains CDR H1, CDR H2, and CDR H3. Each CDR may include amino acid residues from a complementarity determining region as defined by Kabat (i.e. about residues 24-34 (CDR L1), 50-56 (CDR L2) and 89-97 (CDR L3) in the light chain variable region and about residues 31-35 (CDR H1), 50-65 (CDR H2) and 95-102 (CDR H3) in the heavy chain variable region.

As used herein, the term “FcRn” refers a neonatal Fc receptor that binds to the Fc region of an IgG antibody, e.g., an IgG1 antibody. An exemplary FcRn is human FcRn having UniProt ID No. P55899, which is hereby incorporated by reference in its entirety. Human FcRn is believed to be responsible for maintaining the half-life of IgG by binding and trafficking constitutively internalized IgG back to the cell surface for the recycling of IgG.

In some embodiments, the anti-FcRn antibody comprises a heavy chain or light chain. In some embodiments, the anti-FcRN antibody comprises a heavy chain and a light chain in a scFv format. In some embodiments, the heavy and light chain are linked with a peptide linker, such a glycine/serine or glycine/alanine linker.

In some embodiments, the anti-FcRn antibody is M281. In some embodiments, the anti-FcRn antibody is nipocalimab. In some embodiments, M281 and nipocalimab comprise the same amino acid sequence. In some embodiments, M281 and nipocalimab comprise the same heavy chain and light chain amino acid sequence. In some embodiments, M281 and nipocalimab comprise the same variable heavy chain and variable light chain amino acid sequence. As used herein, “M281” and “nipocalimab” refer to the same antibody and can be used interchangeably. In preferred embodiments, the anti-FcRn antibody is M281.

In some embodiments, the anti-FcRn antibody can be M281 (nipocalimab). In some embodiments, nipocalimab comprises or consists of: a light chain comprising or consisting of the sequence:

(SEQ ID NO: 1)
QSALTQPASVSGSPGQSITISCTGTGSDVGSYNLVSWYQQHPGKAPKLMI
YGDSERPSGVSNRFSGSKSGNTASLTISGLQAEDEADYYCSSYAGSGIYV
FGTGTKVTVLGQPKAAPSVTLFPPSSEELQANKATLVCLISDFYPGAVTV
AWKADSSPVKAGVETTTPSKQSNNKYAASSYLSLTPEQWKSHKSYSCQVT
HEGSTVEKTVAPTECS;

and a heavy chain comprises or consists of the sequence:

(SEQ ID NO: 2)
EVQLLESGGGLVQPGGSLRLSCAASGFTFSTYAMGWVRQAPGKGLEWVSS
IGASGSQTRYADSVKGRFTISRDNSKNTLYLQMNSLRAEDTAVYYCARLA
IGDSYWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG.

In some embodiments, the antibody has: (a) a light chain sequence that is at least 95%, 96%, 97% 98%, or 99% identical to SEQ ID NO: 1, wherein the CDR L1 comprises the sequence TGTGSDVGSYNLVS (SEQ ID NO: 3), the CDR L2 comprises the sequence GDSERPS (SEQ ID NO: 4), the CDR L3 comprises the sequence SSYAGSGIYV (SEQ ID NO: 5); and (b) a heavy chain sequence that is at least 95%, 96%, 97% 98%, or 99% identical to SEQ ID NO: 2, wherein the CDR H1 comprises the sequence TYAMG (SEQ ID NO: 6), the CDR H2 comprises the sequence SIGASGSQTRYADS (SEQ ID NO: 7), and the CDR H3 comprises the sequence LAIGDSY (SEQ ID NO: 8). In some embodiments, nipocalimab comprises or consists of a heavy chain comprising or consisting of the sequence set forth in SEQ ID NO: 2, wherein SEQ ID NO: 2 further comprises a C-terminal lysine (K) residue. In some embodiments, SEQ ID NO: 2 comprises a C-terminal lysine (K) residue at position 446. In some embodiments, nipocalimab comprises or consists of a heavy chain comprising or consisting of a sequence lacking a C-terminal lysine (K) residue. In some embodiments, nipocalimab comprises or consists of a heavy chain comprising or consisting of a sequence lacking a C-terminal lysine (K) residue at position 446.

In some embodiments, the antibody heavy chain comprises an amino acid sequence at least 95%, 96%, 97% 98%, or 99% identical to SEQ ID NO: 2 with one or more amino acid substitutions, wherein the amino acid substitutions may be A23V or S30R, relative to the sequence of SEQ ID NO: 2 (according to Kabat numbering). In some embodiments, the antibody light chain comprises an amino acid sequence at least 95%, 96%, 97% 98%, or 99% identical the amino acid sequence of SEQ ID NO: 1 with one or more amino acid substitutions.

In some embodiments, the antibody comprising a light chain variable region comprising an amino acid sequence that is at least 95%, 97%, 99%, or 100% identical to: QSALTQPASVSGSPGQSITISCTGTGSDVGSYNLVSWYQQHPGKAPKLMIYGDSERPSGV SNRFSGSKSGNTASLTISGLQAEDEADYYCSSYAGSGIYVFGTGTKVTVL (SEQ ID NO: 9). In some embodiments, the light chain variable region contains a CDR L1 having the sequence of TGTGSDVGSYNLVS (SEQ ID NO: 3), a CDR L2 having the sequence of GDSERPS (SEQ ID NO: 4), a CDR L3 having the sequence of SSYAGSGIYV (SEQ ID NO: 5). In some embodiments, the antibody comprising a heavy chain variable region comprising an amino acid sequence that is at least 95%, 97%, 99%, or 100% identical to: EVQLLESGGGLVQPGGSLRLSCAASGFTFSTYAMGWVRQAPGKGLEWVSSIGASGSQT RYADSVKGRFTISRDNSKNTLYLQMNSLRAEDTAVYYCARLAIGDSYWGQGTMVTVSS (SEQ ID NO: 10). In some embodiments, the heavy chain variable region contains a CDR H1 having the sequence of TYAMG (SEQ ID NO: 6), a CDR H2 having the sequence of SIGASGSQTRYADS (SEQ ID NO: 7), and a CDR H3 having the sequence of LAIGDSY (SEQ ID NO: 8).

The antibodies may further contain amino acid substitutions, additions, and/or deletions outside of the CDRs (i.e., in framework regions (FRs)). An amino acid substitution, addition, and/or deletion can be a substitution, addition, and/or deletion of one or more amino acids (e.g., 1, 2, 3, 4, 5, 6, 7, 8, or more). An amino acid substitution, addition, and/or deletion can be a substitution, addition, and/or deletion of eight or fewer, seven or fewer, six or fewer, five or fewer, four or fewer, three or fewer, or two or fewer single amino acids. In some embodiments, the antibodies may further include any one or more of the following amino acid substitutions: A23V, S30R, relative to the sequence of any one of SEQ ID NOs: 2, 23-26 (according to Kabat numbering).

In some embodiments, the antibodies may include amino acid substitutions, additions, and/or deletions in the constant regions (e.g., Fc region) of the antibody that, e.g., lead to decreased effector function, e.g., decreased complement-dependent cytolysis (CDC), antibody-dependent cell-mediated cytolysis (ADCC), and/or antibody-dependent cell-mediated phagocytosis (ADCP), and/or decreased B-cell killing. The constant regions are not involved directly in binding an antibody to its target, but exhibit various effector functions, such as participation of the antibody in antibody-dependent cellular toxicity. In some embodiments, the antibodies are characterized by decreased binding (i.e., absence of binding) to human complement factor C1q and/or human Fc receptor on natural killer (NK) cells. In some embodiments, the antibodies are characterized by decreased binding (i.e., absence of binding) to human FcγRI, FcγRIIA, and/or FcγRIIIA. To alter or reduce an antibody-dependent effector function, such as CDC, ADCC, ADCP, and/or B-cell killing, antibodies may be of the IgG class and contain one or more amino acid substitutions E233, L234, G236, D265, D270, N297, E318, K320, K322, A327, A330, P331, and/or P329 (numbering according to the EU System). In some embodiments, the antibodies contain the mutations L234A/L235A or D265A/N297A. In some embodiments, an anti-FcRn antibody is aglycosylated at position 297. The resulting effectorless antibody shows very little binding to complement or Fc receptors (i.e., complement C1q binding), indicating low CDC potential.

In some embodiments, the isolated antibody contains a CDR L1 having the sequence of TGTGSDVGSYNLVS (SEQ ID NO: 3), a CDR L2 having the sequence of GDSERPS (SEQ ID NO: 4), a CDR L3 having the sequence of SSYAGSGIYV (SEQ ID NO: 5), a CDR H1 having the sequence of NYAMG (SEQ ID NO: 12), a CDR H2 having the sequence of SIGASGAQTRYADS (SEQ ID NO: 14), and a CDR H3 having the sequence of LAIGDSY (SEQ ID NO: 8).

In some embodiments, the isolated antibody contains a CDR L1 having the sequence of TGTGSDVGSYNLVS (SEQ ID NO: 3), a CDR L2 having the sequence of GDSERPS (SEQ ID NO: 4), a CDR L3 having the sequence of SSYAGSGIYV (SEQ ID NO: 5), a CDR H1 having the sequence of TYAMG (SEQ ID NO: 4), a CDR H2 having the sequence of SIGASGGQTRYADS (SEQ ID NO: 15), and a CDR H3 having the sequence of LAIGDSY (SEQ ID NO: 8).

In some embodiments, the isolated antibody has a light chain and a heavy chain, wherein the light chain comprises a sequence having the sequence of

(SEQ ID NO: 1)
QSALTQPASVSGSPGQSITISCTGTGSDVGSYNLVSWYQQHPGKAPKLMI
YGDSERPSGVSNRFSGSKSGNTASLTISGLQAEDEADYYCSSYAGSGIYV
FGTGTKVTVLGQPKAAPSVTLFPPSSEELQANKATLVCLISDFYPGAVTV
AWKADSSPVKAGVETTTPSKQSNNKYAASSYLSLTPEQWKSHKSYSCQVT
HEGSTVEKTVAPTECS;

and the heavy chain comprises a sequence having the sequence of

(SEQ ID NO: 2)
EVQLLESGGGLVQPGGSLRLSCAASGFTFSTYAMGWVRQAPGKGLEWVSS
IGASGSQTRYADSVKGRFTISRDNSKNTLYLQMNSLRAEDTAVYYCARLA
IGDSYWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG.

In some embodiments, the isolated antibody has a heavy chain, wherein the heavy chain comprises a sequence having the sequence as set forth in SEQ ID NO: 2, and further comprises a C-terminal lysine (K) residue. In some embodiments, SEQ ID NO: 2 comprises a C-terminal lysine (K) residue at position 446.

In some embodiments, the light chain of the isolated antibody comprises a sequence having at least 90% identity to the sequence of

(SEQ ID NO: 1)
QSALTQPASVSGSPGQSITISCTGTGSDVGSYNLVSWYQQHPGKAPKLMI
YGDSERPSGVSNRFSGSKSGNTASLTISGLQAEDEADYYCSSYAGSGIYV
FGTGTKVTVLGQPKAAPSVTLFPPSSEELQANKATLVCLISDFYPGAVTV
AWKADSSPVKAGVETTTPSKQSNNKYAASSYLSLTPEQWKSHKSYSCQVT
HEGSTVEKTVAPTECS.

In some embodiments, the heavy chain of the isolated antibody comprises a sequence having at least 90% identity to the sequence of

(SEQ ID NO: 2)
EVQLLESGGGLVQPGGSLRLSCAASGFTFSTYAMGWVRQAPGKGLEWVSS
IGASGSQTRYADSVKGRFTISRDNSKNTLYLQMNSLRAEDTAVYYCARLA
IGDSYWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG.

In some embodiments, the isolated antibody has a heavy chain, wherein the heavy chain comprises a sequence having at least 90% identity to the sequence as set forth in SEQ ID NO: 2, and further comprises a C-terminal lysine (K) residue. In some embodiments, the sequence having at least 90% identity to SEQ ID NO: 2 comprises a C-terminal lysine (K) residue at position 446.

In some embodiments, the heavy chain of the isolated antibody comprises a sequence having at least 90% identity to the sequence of

(SEQ ID NO: 23)
EVQLLESGGGLVQPGGSLRLSCAASGFTFSTYAMGWVRQAPGKGLEWVSS
IGSSGAQTRYADSVKGRFTISRDNSKNTLYLQMNSLRAEDTAVYYCARLA
IGDSYWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG.

In some embodiments, the isolated antibody has a heavy chain, wherein the heavy chain comprises a sequence having at least 90% identity to the sequence as set forth in SEQ ID NO: 23, and further comprises a C-terminal lysine (K) residue. In some embodiments, the sequence having at least 90% identity to SEQ ID NO: 23 comprises a C-terminal lysine (K) residue at position 446.

In some embodiments, the heavy chain of the isolated antibody comprises a sequence having at least 90% identity to the sequence of

(SEQ ID NO: 24)
EVQLLESGGGLVQPGGSLRLSCAASGFTFSDYAMGWVRQAPGKGLEWVSS
IGASGSQTRYADSVKGRFTISRDNSKNTLYLQMNSLRAEDTAVYYCARLA
IGDSYWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG.

In some embodiments, the isolated antibody has a heavy chain, wherein the heavy chain comprises a sequence having at least 90% identity to the sequence as set forth in SEQ ID NO: 24, and further comprises a C-terminal lysine (K) residue. In some embodiments, the sequence having at least 90% identity to SEQ ID NO: 24 comprises a C-terminal lysine (K) residue at position 446.

In some embodiments, the heavy chain of the isolated antibody comprises a sequence having at least 90% identity to the sequence of

(SEQ ID NO: 25)
EVQLLESGGGLVQPGGSLRLSCAASGFTFSNYAMGWVRQAPGKGLEWVSS
IGASGAQTRYADSVKGRFTISRDNSKNTLYLQMNSLRAEDTAVYYCARLA
IGDSYWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG.

In some embodiments, the isolated antibody has a heavy chain, wherein the heavy chain comprises a sequence having at least 90% identity to the sequence as set forth in SEQ ID NO: 25, and further comprises a C-terminal lysine (K) residue. In some embodiments, the sequence having at least 90% identity to SEQ ID NO: 25 comprises a C-terminal lysine (K) residue at position 446.

In some embodiments, the heavy chain of the isolated antibody comprises a sequence having at least 90% identity to the sequence of

(SEQ ID NO: 26)
EVQLLESGGGLVQPGGSLRLSCAASGFTFSTYAMGWVRQAPGKGLEWVSS
IGASGGQTRYADSVKGRFTISRDNSKNTLYLQMNSLRAEDTAVYYCARLA
IGDSYWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG.

In some embodiments, the isolated antibody has a heavy chain, wherein the heavy chain comprises a sequence having at least 90% identity to the sequence as set forth in SEQ ID NO: 26, and further comprises a C-terminal lysine (K) residue. In some embodiments, the sequence having at least 90% identity to SEQ ID NO: 26 comprises a C-terminal lysine (K) residue at position 446.

In some embodiments, the isolated antibody has a light chain and a heavy chain, wherein the light chain comprises a sequence having at least 90%, 95%, 98% or 99% identity to the sequence of

(SEQ ID NO: 1)
QSALTQPASVSGSPGQSITISCTGTGSDVGSYNLVSWYQQHPGKAPKLMI
YGDSERPSGVSNRFSGSKSGNTASLTISGLQAEDEADYYCSSYAGSGIYV
FGTGTKVTVLGQPKAAPSVTLFPPSSEELQANKATLVCLISDFYPGAVTV
AWKADSSPVKAGVETTTPSKQSNNKYAASSYLSLTPEQWKSHKSYSCQ
VTHEGSTVEKTVAPTECS;

and the heavy chain comprises a sequence having at least 90%, 95%, 98% or 99% identity to the sequence of

(SEQ ID NO: 2)
EVQLLESGGGLVQPGGSLRLSCAASGFTFSTYAMGWVRQAPGKGLEWVSS
IGASGSQTRYADSVKGRFTISRDNSKNTLYLQMNSLRAEDTAVYYCARLA
IGDSYWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG.

In some embodiments, the isolated antibody has a heavy chain, wherein the heavy chain comprises a sequence having at least 90%, 95%, 98% or 99% identity to the sequence as set forth in SEQ ID NO: 2, and further comprises a C-terminal lysine (K) residue. In some embodiments, the sequence having at least 90%, 95%, 98% or 99% identity to SEQ ID NO: 2 comprises a C-terminal lysine (K) residue at position 446.

In some embodiments, the isolated antibody has a light chain and a heavy chain, wherein the light chain comprises a sequence having at least 90%, 95%, 98% or 99% identity to the sequence of

(SEQ ID NO: 1)
QSALTQPASVSGSPGQSITISCTGTGSDVGSYNLVSWYQQHPGKAPKLMI
YGDSERPSGVSNRFSGSKSGNTASLTISGLQAEDEADYYCSSYAGSGIYV
FGTGTKVTVLGQPKAAPSVTLFPPSSEELQANKATLVCLISDFYPGAVTV
AWKADSSPVKAGVETTTPSKQSNNKYAASSYLSLTPEQWKSHKSYSCQ
VTHEGSTVEKTVAPTECS;

and the heavy chain comprises a sequence having at least 90%, 95%, 98% or 99% identity to the sequence of

(SEQ ID NO: 23)
EVQLLESGGGLVQPGGSLRLSCAASGFTFSTYAMGWVRQAPGKGLEWV
SSIGSSGAQTRYADSVKGRFTISRDNSKNTLYLQMNSLRAEDTAVYYCA
RLAIGDSYWGQGTMVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQ
YASTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPG

In some embodiments, the isolated antibody has a heavy chain, wherein the heavy chain comprises a sequence having at least 90%, 95%, 98% or 99% identity to the sequence as set forth in SEQ ID NO: 23, and further comprises a C-terminal lysine (K) residue. In some embodiments, the sequence having at least 90%, 95%, 98% or 99% identity to SEQ ID NO: 23 comprises a C-terminal lysine (K) residue at position 446.

In some embodiments, the isolated antibody containing has light chain and a heavy chain, wherein the light chain comprises a sequence having at least 90%, 95%, 98% or 99% identity to the sequence of

(SEQ ID NO: 1)
QSALTQPASVSGSPGQSITISCTGTGSDVGSYNLVSWYQQHPGKAPKLMI
YGDSERPSGVSNRFSGSKSGNTASLTISGLQAEDEADYYCSSYAGSGIYV
FGTGTKVTVLGQPKAAPSVTLFPPSSEELQANKATLVCLISDFYPGAVTV
AWKADSSPVKAGVETTTPSKQSNNKYAASSYLSLTPEQWKSHKSYSCQ
VTHEGSTVEKTVAPTECS;

and the heavy chain comprises a sequence having at least 90%, 95%, 98% or 99% identity to the sequence of

(SEQ ID NO: 24)
EVQLLESGGGLVQPGGSLRLSCAASGFTFSDYAMGWVRQAPGKGLEWV
SSIGASGSQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In some embodiments, the isolated antibody has a heavy chain, wherein the heavy chain comprises a sequence having at least 90%, 95%, 98% or 99% identity to the sequence as set forth in SEQ ID NO: 24, and further comprises a C-terminal lysine (K) residue. In some embodiments, the sequence having at least 90%, 95%, 98% or 99% identity to SEQ ID NO: 24 comprises a C-terminal lysine (K) residue at position 446.

In some embodiments, the isolated antibody has a light chain and a heavy chain, wherein the light chain comprises a sequence having at least 90%, 95%, 98% or 99% identity to the sequence of

(SEQ ID NO: 1)
QSALTQPASVSGSPGQSITISCTGTGSDVGSYNLVSWYQQHPGKAPKL
MIYGDSERPSGVSNRFSGSKSGNTASLTISGLQAEDEADYYCSSYAGS
GIYVFGTGTKVTVLGQPKAAPSVTLFPPSSEELQANKATLVCLISDFY
PGAVTVAWKADSSPVKAGVETTTPSKQSNNKYAASSYLSLTPEQWKSH
KSYSCQVTHEGSTVEKTVAPTECS;

and the heavy chain comprises a sequence having at least 90%, 95%, 98% or 99% identity to the sequence of

(SEQ ID NO: 25)
EVQLLESGGGLVQPGGSLRLSCAASGFTFSNYAMGWVRQAPGKGLEWV
SSIGASGAQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In some embodiments, the isolated antibody has a heavy chain, wherein the heavy chain comprises a sequence having at least 90%, 95%, 98% or 99% identity to the sequence as set forth in SEQ ID NO: 25, and further comprises a C-terminal lysine (K) residue. In some embodiments, the sequence having at least 90%, 95%, 98% or 99% identity to SEQ ID NO: 25 comprises a C-terminal lysine (K) residue at position 446.

In some embodiments, the isolated antibody has a light chain and a heavy chain, wherein the light chain comprises a sequence having at least 90%, 95%, 98% or 99% identity to the sequence of

(SEQ ID NO: 1)
QSALTQPASVSGSPGQSITISCTGTGSDVGSYNLVSWYQQHPGKAPKL
MIYGDSERPSGVSNRFSGSKSGNTASLTISGLQAEDEADYYCSSYAGS
GIYVFGTGTKVTVLGQPKAAPSVTLFPPSSEELQANKATLVCLISDFY
PGAVTVAWKADSSPVKAGVETTTPSKQSNNKYAASSYLSLTPEQWKSH
KSYSCQVTHEGSTVEKTVAPTECS;

and the heavy chain comprises a sequence having at least 90%, 95%, 98% or 99% identity to the sequence of

(SEQ ID NO: 26)
EVQLLESGGGLVQPGGSLRLSCAASGFTFSTYAMGWVRQAPGKGLEWV
SSIGASGGQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In some embodiments, the isolated antibody has a heavy chain, wherein the heavy chain comprises a sequence having at least 90%, 95%, 98% or 99% identity to the sequence as set forth in SEQ ID NO: 26, and further comprises a C-terminal lysine (K) residue. In some embodiments, the sequence having at least 90%, 95%, 98% or 99% identity to SEQ ID NO: 26 comprises a C-terminal lysine (K) residue at position 446.

In some embodiments, the heavy chain of the isolated antibody comprises a sequence having at least 95%, 97%, 99%, or 100% identity to the sequence of any one of SEQ ID NOs: 2, 23-26. In some embodiments, the heavy chain of the isolated antibody comprises a sequence having at least 95%, 97%, 99%, or 100% identity to the sequence of any one of SEQ ID NOs: 2, 23-26 further comprising a C-terminal lysine (K) residue at position 446 of any one of SEQ ID NOs: 2, 23-26. In some embodiments, the light chain of the isolated antibody comprises a sequence having at least 95%, 97%, 99%, or 100% identity to the sequence of SEQ ID NO: 1.

In some embodiments, the heavy chain of the isolated antibody comprises a sequence having no more than 5, 4, 3, 2 or 1 single amino acid substitutions relative to the amino acid sequence of any one of SEQ ID NOs: 2. In some embodiments, the heavy chain of the isolated antibody comprises a sequence having no more than 5, 4, 3, 2 or 1 single amino acid substitutions relative to the amino acid sequence of any one of SEQ ID NOs: 2 and further comprises a C-terminal lysine (K) residue at position 446. In some embodiments, the light chain of the isolated antibody comprises a sequence having no more than 5, 4, 3, 2 or 1 single amino acid substitutions relative to the sequence of SEQ ID NO: 1.

In some embodiments, the isolated antibody further includes any one or more of the following amino acid substitutions: A23V, S30R (According to EU Numbering), relative to the sequence of any one of SEQ ID NOs: 2, 23-26. In some embodiments, the isolated antibody further includes any one or more of the following amino acid substitutions: A23V, S30R (According to EU Numbering), relative to the sequence of any one of SEQ ID NOs: 2, 23-26 and further comprises a C-terminal lysine (K) residue at position 446 of any one of SEQ ID NOs: 2, 23-26.

In some embodiment, the isolated antibody does not contain a C-terminal lysine at residue 446, relative to the sequence of any one of SEQ ID NO: 2.

In some embodiments, the isolated antibody has a light chain and a heavy chain, wherein the light chain comprises or consists of the sequence of

(SEQ ID NO: 1)
QSALTQPASVSGSPGQSITISCTGTGSDVGSYNLVSWYQQHPGKAPKL
MIYGDSERPSGVSNRFSGSKSGNTASLTISGLQAEDEADYYCSSYAGS
GIYVFGTGTKVTVLGQPKAAPSVTLFPPSSEELQANKATLVCLISDFY
PGAVTVAWKADSSPVKAGVETTTPSKQSNNKYAASSYLSLTPEQWKSH
KSYSCQVTHEGSTVEKTVAPTECS;

and the heavy chain comprises or consists of the sequence of

(SEQ ID NO: 2)
EVQLLESGGGLVQPGGSLRLSCAASGFTFSTYAMGWVRQAPGKGLEWV
SSIGASGSQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In some embodiments, the isolated antibody has a light chain and a heavy chain, wherein the light chain comprises or consists of the sequence of

(SEQ ID NO: 1)
QSALTQPASVSGSPGQSITISCTGTGSDVGSYNLVSWYQQHPGKAPKL
MIYGDSERPSGVSNRFSGSKSGNTASLTISGLQAEDEADYYCSSYAGS
GIYVFGTGTKVTVLGQPKAAPSVTLFPPSSEELQANKATLVCLISDFY
PGAVTVAWKADSSPVKAGVETTTPSKQSNNKYAASSYLSLTPEQWKSH
KSYSCQVTHEGSTVEKTVAPTECS;

and the heavy chain comprises or consists of the sequence of

(SEQ ID NO: 23)
EVQLLESGGGLVQPGGSLRLSCAASGFTFSTYAMGWVRQAPGKGLEWV
SSIGSSGAQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In some embodiments, the isolated antibody has a light chain and a heavy chain, wherein the light chain comprises or consists of the sequence of

(SEQ ID NO: 1)
QSALTQPASVSGSPGQSITISCTGTGSDVGSYNLVSWYQQHPGKAPKL
MIYGDSERPSGVSNRFSGSKSGNTASLTISGLQAEDEADYYCSSYAGS
GIYVFGTGTKVTVLGQPKAAPSVTLFPPSSEELQANKATLVCLISDFY
PGAVTVAWKADSSPVKAGVETTTPSKQSNNKYAASSYLSLTPEQWKSH
KSYSCQVTHEGSTVEKTVAPTECS;

and the heavy chain comprises or consists of the sequence of

(SEQ ID NO: 24)
EVQLLESGGGLVQPGGSLRLSCAASGFTFSDYAMGWVRQAPGKGLEWV
SSIGASGSQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In some embodiments, the isolated antibody has a light chain and a heavy chain, wherein the light chain comprises the sequence of

(SEQ ID NO: 1)
QSALTQPASVSGSPGQSITISCTGTGSDVGSYNLVSWYQQHPGKAPKL
MIYGDSERPSGVSNRFSGSKSGNTASLTISGLQAEDEADYYCSSYAGS
GIYVFGTGTKVTVLGQPKAAPSVTLFPPSSEELQANKATLVCLISDFY
PGAVTVAWKADSSPVKAGVETTTPSKQSNNKYAASSYLSLTPEQWKSH
KSYSCQVTHEGSTVEKTVAPTECS;

and the heavy chain comprises or consists of the sequence of

(SEQ ID NO: 25)
EVQLLESGGGLVQPGGSLRLSCAASGFTFSNYAMGWVRQAPGKGLEWV
SSIGASGAQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In some embodiments, the isolated antibody has a light chain and a heavy chain, wherein the light chain comprises or consists of the sequence of

(SEQ ID NO: 1)
QSALTQPASVSGSPGQSITISCTGTGSDVGSYNLVSWYQQHPGKAPKL
MIYGDSERPSGVSNRFSGSKSGNTASLTISGLQAEDEADYYCSSYAGS
GIYVFGTGTKVTVLGQPKAAPSVTLFPPSSEELQANKATLVCLISDFY
PGAVTVAWKADSSPVKAGVETTTPSKQSNNKYAASSYLSLTPEQWKSH
KSYSCQVTHEGSTVEKTVAPTECS;

and the heavy chain comprises or consists of the sequence of

(SEQ ID NO: 26)
EVQLLESGGGLVQPGGSLRLSCAASGFTFSTYAMGWVRQAPGKGLEWV
SSIGASGGQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

As used herein, the term “percent (%) identity” refers to the percentage of amino acid (or nucleic acid) residues of a candidate sequence, e.g., an anti-FcRn antibody of the disclosure, that are identical to the amino acid (or nucleic acid) residues of a reference sequence, e.g., a wild-type anti-FcRn antibody, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

100×(fraction of A/B)
where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In some embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position. A position may be altered by a substitution, deletion, or insertion. A substitution, deletion, or insertion may comprise a certain number of amino acids, (e.g., 1, 2, 3, 4, 5, 6, 7, 8, 9, 10, 11, 12, 13, 14, 15, or more). When describing a substitution, deletion, or insertion of no more than “n” amino acids, this is meant that the substitution, deletion, or insertion comprises, e.g., 1, 2, 3, 4, 5, 6, 7, 8, 9, 10, 11, 12, 13, 14, 15, or “n” amino acids. The number or substitutions, deletions, or insertions can comprise a percent of the total sequence (e.g., 1%, 5%, 10%, 15%, 20%, or more) where the number of substitutions, deletions, or insertions alters 5%, 10%, 15%, 20% or more, of the amino acids in the total sequence.

In some embodiments, the Fc domain of the antibody is not fucosylated. In some embodiments of all the methods described herein, the Fc domain of the antibody is not glycosylated. In some embodiments of all the methods described herein, the antibody lacks effector function. In some embodiments of all the methods described herein, the antibody is an IgG1 antibody.

In some embodiments, the anti-FcRn antibody nipocalimab, RVT-1401 (HL161), rozanolixizumab (UCB7665), ALXN1830, ABY-039, or efgartigimod. RVT-1401 (also referred to as HL161BKN) is described in PCT Pub. No. WO2020097099, rozanolixizumab is described in PCT Pub. No. WO2014019727, and efgartigimod (ARGX-113) is described in PCT Pub. No. WO2015100299, each of which is hereby incorporated by reference in its entirety. In some embodiments, the anti-FcRn antibody is a biosimilar of any of anti-FcRn antibodies provided for herein.

In some embodiments, the methods described herein comprise administering the anti-FcRn antibody to the pediatric subject or patient. The terms “pediatric subject” or “pediatric patient” can be used interchangeably. In some embodiments, a pediatric subject is 2 years old to less than 18 years old. In some embodiments, a pediatric subject is 2 years old to less than 12 years old. In some embodiments, a pediatric subject is 12 years old to less than 18 years old. In some embodiments, a pediatric subject is an adolescent, defined as less than 18 years old or 16 years old to 17 years old. In some embodiments, the pediatric subject is a teenager, defined as 12 years old to 15 years old. In some embodiments, the pediatric subject is a child, defined as 2 years old to 11 years old.

Myasthenia gravis in the pediatric population is classified into three categories: 1) transient neonatal myasthenia, 2) juvenile myasthenia gravis (juvenile MG) and 3) congenital myasthenic syndromes (CMS). Juvenile MG is an autoimmune disorder in which autoantibodies to structural components of the neuromuscular junction disrupt neuromuscular transmission. In some embodiments, the methods described herein comprise administering the anti-FcRn antibody to the pediatric subject diagnosed with transient neonatal myasthenia. In some embodiments, the methods described herein comprise administering the anti-FcRn antibody to the pediatric subject diagnosed with juvenile myasthenia gravis (juvenile MG). In some embodiments, the methods described herein comprise administering the anti-FcRn antibody to the pediatric subject diagnosed with congenital myasthenic syndromes (CMS).

In some embodiments, a method of treating pediatric myasthenia gravis in a pediatric patient in need thereof, is provided. In some embodiments, the method comprises administering an initial loading dose of about 30 mg/kg to about 60 mg/kg of an anti-FcRn antibody followed by administering a maintenance dose of about 15 mg/kg to about 30 mg/kg of the anti-FcRn antibody, wherein the anti-FcRn antibody comprises: a heavy chain comprising a HCDR1 of SEQ ID NO: 6, a HCDR2 of SEQ ID NO: 7, and a HCDR3 of SEQ ID NO: 8; and a light chain comprising a LCDR1 of SEQ ID NO: 3, a LCDR2 of SEQ ID NO: 4, and a LCDR3 of SEQ ID NO: 5; wherein the administration reduces serum IgG in the patient by at least 10%, at least 20%, at least 30%, at least 40%, at least 50%, at least 60%, at least 70%, at least 80%, or at least 90% of baseline serum IgG, and wherein the pediatric myasthenia gravis is selected from transient neonatal myasthenia, juvenile myasthenia gravis, congenital myasthenia gravis syndrome, or any combination thereof.

In some embodiments, a method of treating pediatric myasthenia gravis in a pediatric patient in need thereof, is provided. In some embodiments, the method comprises administering an initial loading dose of about 30 mg/kg to about 60 mg/kg of an anti-FcRn antibody followed by administering a maintenance dose of about 15 mg/kg to about 30 mg/kg of the anti-FcRn antibody, wherein the anti-FcRn antibody comprises: a heavy chain comprising a HCDR1 of SEQ ID NO: 6, a HCDR2 of SEQ ID NO: 7, and a HCDR3 of SEQ ID NO: 8; and a light chain comprising a LCDR1 of SEQ ID NO: 3, a LCDR2 of SEQ ID NO: 4, and a LCDR3 of SEQ ID NO: 5; wherein the administration reduces serum IgG in the patient by at least 10%, at least 20%, at least 30%, at least 40%, at least 50%, at least 60%, at least 70%, at least 80%, or at least 90% of baseline serum IgG, and wherein the pediatric myasthenia gravis is transient neonatal myasthenia.

In some embodiments, a method of treating pediatric myasthenia gravis in a pediatric patient in need thereof, is provided. In some embodiments, the method comprises administering an initial loading dose of about 30 mg/kg to about 60 mg/kg of an anti-FcRn antibody followed by administering a maintenance dose of about 15 mg/kg to about 30 mg/kg of the anti-FcRn antibody, wherein the anti-FcRn antibody comprises: a heavy chain comprising a HCDR1 of SEQ ID NO: 6, a HCDR2 of SEQ ID NO: 7, and a HCDR3 of SEQ ID NO: 8; and a light chain comprising a LCDR1 of SEQ ID NO: 3, a LCDR2 of SEQ ID NO: 4, and a LCDR3 of SEQ ID NO: 5; wherein the administration reduces serum IgG in the patient by at least 10%, at least 20%, at least 30%, at least 40%, at least 50%, at least 60%, at least 70%, at least 80%, or at least 90% of baseline serum IgG, and wherein the pediatric myasthenia gravis is juvenile myasthenia gravis.

In some embodiments, a method of treating pediatric myasthenia gravis in a pediatric patient in need thereof, is provided. In some embodiments, the method comprises administering an initial loading dose of about 30 mg/kg to about 60 mg/kg of an anti-FcRn antibody followed by administering a maintenance dose of about 15 mg/kg to about 30 mg/kg of the anti-FcRn antibody, wherein the anti-FcRn antibody comprises: a heavy chain comprising a HCDR1 of SEQ ID NO: 6, a HCDR2 of SEQ ID NO: 7, and a HCDR3 of SEQ ID NO: 8; and a light chain comprising a LCDR1 of SEQ ID NO: 3, a LCDR2 of SEQ ID NO: 4, and a LCDR3 of SEQ ID NO: 5; wherein the administration reduces serum IgG in the patient by at least 10%, at least 20%, at least 30%, at least 40%, at least 50%, at least 60%, at least 70%, at least 80%, or at least 90% of baseline serum IgG, and wherein the pediatric myasthenia gravis congenital myasthenia gravis syndrome.

In some embodiments, the antibody is administered as part of a pharmaceutical composition comprising 5-60 mg/ml of the antibody. In some embodiments, the antibody is administered at an initial dose that is 60 mg/kg. In some embodiments, the antibody is administered at an initial dose that is 30 mg/kg. In some embodiments, the antibody is administered at an initial loading dose that is 60 mg/kg. In some embodiments, the antibody is administered at an initial loading dose that is 30 mg/kg. In some embodiments, the antibody is administered at a dose from about 5 mg/kg to about 60 mg/kg, about 5 mg/kg to about 15 mg/kg, about 15 mg/kg to about 60 mg/kg, or about 30 mg/kg to about 60 mg/kg. In some embodiments, the antibody is administered at a dose of about 5 mg/kg, about 15 mg/kg, about 30 mg/kg, about 45 mg/kg, or about 60 mg/kg. In some embodiments, the antibody is administered at a dose of, or about, 5, 10, 15, 20, 25, 30, 35, 40, 45, 50, 55, or 60 mg/kg. In some embodiments, the antibody is administered at a single dose, or at an initial loading dose and a maintenance dose. In some embodiments, the initial loading dose and the maintenance dose are at the same dose. In some embodiments, the loading dose and the maintenance dose are not the same dose. In some embodiments, the loading dose is administered at a dose from about 5 mg/kg to about 60 mg/kg, about 5 mg/kg to about 15 mg/kg, about 15 mg/kg to about 60 mg/kg, or about 30 mg/kg to about 60 mg/kg. In some embodiments, the loading dose is administered at a dose of 60 mg/kg. In some embodiments, the loading dose is administered at a dose of 30 mg/kg. In some embodiments, the maintenance dose is administered at a dose from about 5 mg/kg to about 60 mg/kg, about 5 mg/kg to about 15 mg/kg, about 15 mg/kg to about 60 mg/kg, or about 30 mg/kg to about 60 mg/kg. In some embodiments, the maintenance dose is administered at a dose of about 5 mg/kg, about 15 mg/kg, about 30 mg/kg, about 45 mg/kg, or about 60 mg/kg. In some embodiments, the maintenance dose is administered at a dose of 5, 10, 15, 20, 25, 30, 35, 40, 45, 50, 55, or 60 mg/kg. In some embodiments, the maintenance dose is administered at a dose of 15 mg/kg. In some embodiments, the loading dose is administered at a dose of about 30 mg/kg and a maintenance dose of about 15 mg/kg. In some embodiments, the loading dose is administered at a dose of 30 mg/kg and a maintenance dose of 15 mg/kg.

In some embodiments, the antibody is administered weekly, once every two weeks, or monthly. In some embodiments, the initial dose is different from the weekly or once every two weeks dose. In some embodiments, the dose is the same every time it is administered. In some embodiments, the antibody is administered at least every week, every two weeks, every 3 weeks, or every four weeks (i.e., once a month). In some embodiments, a first infusion of the antibody is administered at a loading dose of about 60 mg/kg and a second infusion of the antibody is administered weekly at a maintenance dose of about 15 mg/kg. In some embodiments, a first infusion of the antibody is administered at a loading dose of about 30 mg/kg and a second infusion of the antibody is administered weekly at a maintenance dose of about 15 mg/kg. In some embodiments, a first infusion of the antibody is administered at a loading dose of about 60 mg/kg and a second infusion of the antibody is administered every 2 weeks at a maintenance dose of about 15 mg/kg. In some embodiments, a first infusion of the antibody is administered at a loading dose of about 30 mg/kg and a second infusion of the antibody is administered every 2 weeks at a maintenance dose of about 15 mg/kg. In some embodiments, a first infusion of the antibody is administered at a loading dose of about 60 mg/kg and a second infusion of the antibody is administered every 3 weeks at a maintenance dose of about 15 mg/kg. In some embodiments, a first infusion of the antibody is administered at a loading dose of about 30 mg/kg and a second infusion of the antibody is administered every 3 weeks at a maintenance dose of about 15 mg/kg. In some embodiments, a first infusion of the antibody is administered at a loading dose of about 60 mg/kg and a second infusion of the antibody is administered every 4 weeks at a maintenance dose of about 15 mg/kg. In some embodiments, a first infusion of the antibody is administered at a loading dose of about 30 mg/kg and a second infusion of the antibody is administered every 4 weeks at a maintenance dose of about 15 mg/kg. In some embodiments, a first infusion of the antibody is administered at a loading dose of about 60 mg/kg and a second infusion of the antibody is administered every month at a maintenance dose of about 15 mg/kg. In some embodiments, a first infusion of the antibody is administered at a loading dose of about 30 mg/kg and a second infusion of the antibody is administered every month at a maintenance dose of about 15 mg/kg.

In some embodiments, a first infusion of the antibody is administered at a loading dose of about 5-60 mg/kg and a second infusion of the antibody is administered weekly at a maintenance dose of about 5-60 mg/kg. In some embodiments, a first infusion of the antibody is administered at a loading dose of about 5-60 mg/kg and a second infusion of the antibody is administered every 2 weeks at a maintenance dose of about 5-60 mg/kg. In some embodiments, a first infusion of the antibody is administered at a loading dose of about 5-60 mg/kg and a second infusion of the antibody is administered every 3 weeks at a maintenance dose of about 5-60 mg/kg. In some embodiments, a first infusion of the antibody is administered at a loading dose of about 5-60 mg/kg and a second infusion of the antibody is administered every 4 weeks at a maintenance dose of about 5-60 mg/kg. In some embodiments, a first infusion of the antibody is administered at a loading dose of about 5-60 mg/kg and a second infusion of the antibody is administered every month at a maintenance dose of about 5-60 mg/kg. In some embodiments, a first infusion of the antibody is administered at a loading dose of about 5-30 mg/kg and a second infusion of the antibody is administered weekly at a maintenance dose of about 5-30 mg/kg. In some embodiments, a first infusion of the antibody is administered at a loading dose of about 5-30 mg/kg and a second infusion of the antibody is administered every 2 weeks at a maintenance dose of about 5-30 mg/kg. In some embodiments, a first infusion of the antibody is administered at a loading dose of about 5-30 mg/kg and a second infusion of the antibody is administered every 3 weeks at a maintenance dose of about 5-30 mg/kg. In some embodiments, a first infusion of the antibody is administered at a loading dose of about 5-30 mg/kg and a second infusion of the antibody is administered every 4 weeks at a maintenance dose of about 5-30 mg/kg. In some embodiments, a first infusion of the antibody is administered at a loading dose of about 5-30 mg/kg and a second infusion of the antibody is administered every month at a maintenance dose of about 5-30 mg/kg. In some embodiments, a first infusion of the antibody is administered at a loading dose of about 15-30 mg/kg and a second infusion of the antibody is administered weekly at a maintenance dose of about 15-30 mg/kg. In some embodiments, a first infusion of the antibody is administered at a loading dose of about 15-30 mg/kg and a second infusion of the antibody is administered every 2 weeks at a maintenance dose of about 15-30 mg/kg. In some embodiments, a first infusion of the antibody is administered at a loading dose of about 15-30 mg/kg and a second infusion of the antibody is administered every 3 weeks at a maintenance dose of about 15-30 mg/kg. In some embodiments, a first infusion of the antibody is administered at a loading dose of about 15-30 mg/kg and a second infusion of the antibody is administered every 4 weeks at a maintenance dose of about 15-30 mg/kg. In some embodiments, a first infusion of the antibody is administered at a loading dose of about 15-30 mg/kg and a second infusion of the antibody is administered every month at a maintenance dose of about 15-30 mg/kg. In some embodiments, a first infusion of the antibody is administered at a loading dose of about 60 mg/kg and a second infusion of the antibody is administered weekly at a maintenance dose of about 30 mg/kg. In some embodiments, a first infusion of the antibody is administered at a loading dose of about 60 mg/kg and a second infusion of the antibody is administered every 2 weeks at a maintenance dose of about 30 mg/kg. In some embodiments, a first infusion of the antibody is administered at a loading dose of about 60 mg/kg and a second infusion of the antibody is administered every 3 weeks at a maintenance dose of about 30 mg/kg. In some embodiments, a first infusion of the antibody is administered at a loading dose of about 60 mg/kg and a second infusion of the antibody is administered every 4 weeks at a maintenance dose of about 30 mg/kg. In some embodiments, a first infusion of the antibody is administered at a loading dose of about 60 mg/kg and a second infusion of the antibody is administered every month at a maintenance dose of about 30 mg/kg. In some embodiments, a first infusion of the antibody is administered at a loading dose of about 30 mg/kg and a second infusion of the antibody is administered weekly at a maintenance dose of about 30 mg/kg. In some embodiments, a first infusion of the antibody is administered at a loading dose of about 30 mg/kg and a second infusion of the antibody is administered every 2 weeks at a maintenance dose of about 30 mg/kg. In some embodiments, a first infusion of the antibody is administered at a loading dose of about 30 mg/kg and a second infusion of the antibody is administered every 3 weeks at a maintenance dose of about 30 mg/kg. In some embodiments, a first infusion of the antibody is administered at a loading dose of about 30 mg/kg and a second infusion of the antibody is administered every 4 weeks at a maintenance dose of about 30 mg/kg. In some embodiments, a first infusion of the antibody is administered at a loading dose of about 30 mg/kg and a second infusion of the antibody is administered every month at a maintenance dose of about 30 mg/kg. In some embodiments, additional infusions are performed at a maintenance dose of about 5-60 mg/kg. In some embodiments, additional infusions are performed at a maintenance dose of about 5-30 mg/kg. In some embodiments, additional infusions are performed at a maintenance dose of about 15-30 mg/kg. In some embodiments, additional infusions are performed at a maintenance dose of about 15 mg/kg. In some embodiments, additional infusions are performed at a maintenance dose of about 30 mg/kg. In some embodiments, the infusions (including the first, second, and/or additional) are administered every two weeks, every 3 weeks, every 4 weeks, or every month. In some embodiments, the method includes an initial (loading) dose followed by a biweekly (e.g., every two weeks) maintenance dose. In some embodiments, the method includes an initial (loading) dose followed by an every 3 weeks maintenance dose. In some embodiments, the method includes an initial (loading) dose followed by an every 4 weeks maintenance dose. In some embodiments, the method includes an initial (loading) dose followed by an every month maintenance dose. In some embodiments, the initial dose is higher than the biweekly maintenance dose. In some embodiments, the initial dose is the same as the biweekly maintenance dose.

In some embodiments, the administration of the antibody or pharmaceutical composition takes place over about 30-90 minutes. In some embodiments, the administration takes place over about 15-60 minutes. In some embodiments, the administration takes place in about 15 to about 30 minutes. In some embodiments, the administration takes place in about 15 to about 45 minutes. In some embodiments, the administration takes place in about 15 to about 90 minutes. In some embodiments, the administration takes place in about 15 to about 120 minutes. In some embodiments, the administration takes place in about 15 minutes, about 30 minutes, about 45 minutes, about 60 minutes, about 90 minutes, or about 120 minutes.

In some embodiments, the composition is administered parenterally. In some embodiments, the composition is administered intravenously or subcutaneously. In some embodiments, the composition is administered intraperitoneal, intradermally, or intramuscularly. In some embodiments, the composition is administered intravenously. In some embodiments, the composition is administered subcutaneously. In some embodiments, the composition is administered using an infusion pump. In some embodiments, the composition administered using an autoinjector. In some embodiments, the composition is administered using a patch pump injector. In some embodiments, the composition is administered using a wearable injector. In some embodiments, the composition is administered using a Sorrel™ pump. In some embodiments, the composition is administered using a pump, such as those in U.S. Pat. No. 9,943,642, which is hereby incorporated in its entirety.

In certain embodiments, the method of treating generalized myasthenia gravis in a pediatric subject in need thereof comprises administering a therapeutically effective amount of nipocalimab intravenously to the pediatric subject, wherein the therapeutically effective amount of nipocalimab is 5-60 mg/kg. In certain embodiments, the method of treating generalized myasthenia gravis in a pediatric subject in need thereof comprises administering a therapeutically effective amount of nipocalimab intravenously to the pediatric subject, wherein the therapeutically effective amount of nipocalimab is 5-30 mg/kg. In certain embodiments, the method of treating generalized myasthenia gravis in a pediatric subject in need thereof comprises administering a therapeutically effective amount of nipocalimab intravenously to the pediatric subject, wherein the therapeutically effective amount of nipocalimab is 15-30 mg/kg. In certain embodiments, the method of treating generalized myasthenia gravis in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 2 weeks. In certain embodiments, the method of treating generalized myasthenia gravis in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 2 weeks. In certain embodiments, the method of treating generalized myasthenia gravis in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 2 weeks. In certain embodiments, the method of treating generalized myasthenia gravis in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 4 weeks. In certain embodiments, the method of treating generalized myasthenia gravis in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 4 weeks. In certain embodiments, the method of treating generalized myasthenia gravis in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 4 weeks. In certain embodiments, the method of treating generalized myasthenia gravis in a pediatric subject in need thereof comprises administering a therapeutically effective amount of nipocalimab intravenously to the pediatric subject, wherein the therapeutically effective amount of nipocalimab is 30 mg/kg. In certain embodiments, the method of treating generalized myasthenia gravis in a pediatric subject in need thereof comprises administering a therapeutically effective amount of nipocalimab intravenously to the pediatric subject, wherein the therapeutically effective amount of nipocalimab is 15 mg/kg. In certain embodiments, the method of treating generalized myasthenia gravis in a pediatric subject in need thereof comprises administering nipocalimab intravenously to the pediatric subject, wherein the nipocalimab is administered as a loading dose at 30 mg/kg followed by a maintenance dose at 15 mg/kg every 2 weeks. In certain embodiments, the method of treating generalized myasthenia gravis in a pediatric subject in need thereof comprises administering nipocalimab intravenously to the pediatric subject, wherein the nipocalimab is administered as a loading dose at 30 mg/kg followed by a maintenance dose at 30 mg/kg every 4 weeks.

In certain embodiments, the method of treating transient neonatal myasthenia in a pediatric subject in need thereof comprises administering a therapeutically effective amount of nipocalimab intravenously to the pediatric subject, wherein the therapeutically effective amount of nipocalimab is 5-60 mg/kg. In certain embodiments, the method of treating transient neonatal myasthenia in a pediatric subject in need thereof comprises administering a therapeutically effective amount of nipocalimab intravenously to the pediatric subject, wherein the therapeutically effective amount of nipocalimab is 5-30 mg/kg. In certain embodiments, the method of treating transient neonatal myasthenia in a pediatric subject in need thereof comprises administering a therapeutically effective amount of nipocalimab intravenously to the pediatric subject, wherein the therapeutically effective amount of nipocalimab is 15-30 mg/kg. In certain embodiments, the method of treating transient neonatal myasthenia in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 2 weeks. In certain embodiments, the method of treating transient neonatal myasthenia in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 2 weeks. In certain embodiments, the method of treating transient neonatal myasthenia in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 2 weeks. In certain embodiments, the method of treating transient neonatal myasthenia in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 4 weeks. In certain embodiments, the method of treating transient neonatal myasthenia in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 4 weeks. In certain embodiments, the method of treating transient neonatal myasthenia in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 4 weeks. In certain embodiments, the method of treating transient neonatal myasthenia in a pediatric subject in need thereof comprises administering a therapeutically effective amount of nipocalimab intravenously to the pediatric subject, wherein the therapeutically effective amount of nipocalimab is 30 mg/kg. In certain embodiments, the method of treating transient neonatal myasthenia in a pediatric subject in need thereof comprises administering a therapeutically effective amount of nipocalimab intravenously to the pediatric subject, wherein the therapeutically effective amount of nipocalimab is 15 mg/kg. In certain embodiments, the method of treating transient neonatal myasthenia in a pediatric subject in need thereof comprises administering nipocalimab intravenously to the pediatric subject, wherein the nipocalimab is administered as a loading dose at 30 mg/kg followed by a maintenance dose at 15 mg/kg every 2 weeks. In certain embodiments, the method of treating transient neonatal myasthenia in a pediatric subject in need thereof comprises administering nipocalimab intravenously to the pediatric subject, wherein the nipocalimab is administered as a loading dose at 30 mg/kg followed by a maintenance dose at 30 mg/kg every 4 weeks.

In certain embodiments, the method of treating juvenile myasthenia gravis in a pediatric subject in need thereof comprises administering a therapeutically effective amount of nipocalimab intravenously to the pediatric subject, wherein the therapeutically effective amount of nipocalimab is 5-60 mg/kg. In certain embodiments, the method of treating juvenile myasthenia gravis in a pediatric subject in need thereof comprises administering a therapeutically effective amount of nipocalimab intravenously to the pediatric subject, wherein the therapeutically effective amount of nipocalimab is 5-30 mg/kg. In certain embodiments, the method of treating juvenile myasthenia gravis in a pediatric subject in need thereof comprises administering a therapeutically effective amount of nipocalimab intravenously to the pediatric subject, wherein the therapeutically effective amount of nipocalimab is 15-30 mg/kg. In certain embodiments, the method of treating juvenile myasthenia gravis in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 2 weeks. In certain embodiments, the method of treating juvenile myasthenia gravis in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 2 weeks. In certain embodiments, the method of treating juvenile myasthenia gravis in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 2 weeks. In certain embodiments, the method of treating juvenile myasthenia gravis in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 4 weeks. In certain embodiments, the method of treating juvenile myasthenia gravis in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 4 weeks. In certain embodiments, the method of treating juvenile myasthenia gravis in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 4 weeks. In certain embodiments, the method of treating juvenile myasthenia gravis in a pediatric subject in need thereof comprises administering a therapeutically effective amount of nipocalimab intravenously to the pediatric subject, wherein the therapeutically effective amount of nipocalimab is 30 mg/kg. In certain embodiments, the method of treating juvenile myasthenia gravis in a pediatric subject in need thereof comprises administering a therapeutically effective amount of nipocalimab intravenously to the pediatric subject, wherein the therapeutically effective amount of nipocalimab is 15 mg/kg. In certain embodiments, the method of treating juvenile myasthenia gravis in a pediatric subject in need thereof comprises administering nipocalimab intravenously to the pediatric subject, wherein the nipocalimab is administered as a loading dose at 30 mg/kg followed by a maintenance dose at 15 mg/kg every 2 weeks. In certain embodiments, the method of treating juvenile myasthenia gravis in a pediatric subject in need thereof comprises administering nipocalimab intravenously to the pediatric subject, wherein the nipocalimab is administered as a loading dose at 30 mg/kg followed by a maintenance dose at 30 mg/kg every 4 weeks.

In certain embodiments, the method of treating congenital myasthenic syndromes (CMS) in a pediatric subject in need thereof comprises administering a therapeutically effective amount of nipocalimab intravenously to the pediatric subject, wherein the therapeutically effective amount of nipocalimab is 5-60 mg/kg. In certain embodiments, the method of treating congenital myasthenic syndromes (CMS) in a pediatric subject in need thereof comprises administering a therapeutically effective amount of nipocalimab intravenously to the pediatric subject, wherein the therapeutically effective amount of nipocalimab is 5-30 mg/kg. In certain embodiments, the method of treating congenital myasthenic syndromes (CMS) in a pediatric subject in need thereof comprises administering a therapeutically effective amount of nipocalimab intravenously to the pediatric subject, wherein the therapeutically effective amount of nipocalimab is 15-30 mg/kg. In certain embodiments, the method of treating congenital myasthenic syndromes (CMS) in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 2 weeks. In certain embodiments, the method of treating congenital myasthenic syndromes (CMS) in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 2 weeks. In certain embodiments, the method of treating congenital myasthenic syndromes (CMS) in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 2 weeks. In certain embodiments, the method of treating congenital myasthenic syndromes (CMS) in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 4 weeks. In certain embodiments, the method of treating congenital myasthenic syndromes (CMS) in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 4 weeks. In certain embodiments, the method of treating congenital myasthenic syndromes (CMS) in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 4 weeks. In certain embodiments, the method of treating congenital myasthenic syndromes (CMS) in a pediatric subject in need thereof comprises administering a therapeutically effective amount of nipocalimab intravenously to the pediatric subject, wherein the therapeutically effective amount of nipocalimab is 30 mg/kg. In certain embodiments, the method of treating congenital myasthenic syndromes (CMS) in a pediatric subject in need thereof comprises administering a therapeutically effective amount of nipocalimab intravenously to the pediatric subject, wherein the therapeutically effective amount of nipocalimab is 15 mg/kg. In certain embodiments, the method of treating congenital myasthenic syndromes (CMS) in a pediatric subject in need thereof comprises administering nipocalimab intravenously to the pediatric subject, wherein the nipocalimab is administered as a loading dose at 30 mg/kg followed by a maintenance dose at 15 mg/kg every 2 weeks. In certain embodiments, the method of treating congenital myasthenic syndromes (CMS) in a pediatric subject in need thereof comprises administering nipocalimab intravenously to the pediatric subject, wherein the nipocalimab is administered as a loading dose at 30 mg/kg followed by a maintenance dose at 30 mg/kg every 4 weeks.

In certain embodiments, the method of treating transient neonatal myasthenia, juvenile myasthenia gravis, congenital myasthenia gravis syndromes (CMS), or any combination thereof, in a pediatric subject in need thereof comprises administering a therapeutically effective amount of nipocalimab intravenously to the pediatric subject, wherein the therapeutically effective amount of nipocalimab is 5-60 mg/kg. In certain embodiments, the method of treating transient neonatal myasthenia, juvenile myasthenia gravis, congenital myasthenia gravis syndromes (CMS), or any combination thereof, in a pediatric subject in need thereof comprises administering a therapeutically effective amount of nipocalimab intravenously to the pediatric subject, wherein the therapeutically effective amount of nipocalimab is 5-30 mg/kg. In certain embodiments, the method of treating transient neonatal myasthenia, juvenile myasthenia gravis, congenital myasthenia gravis syndromes (CMS), or any combination thereof, in a pediatric subject in need thereof comprises administering a therapeutically effective amount of nipocalimab intravenously to the pediatric subject, wherein the therapeutically effective amount of nipocalimab is 15-30 mg/kg. In certain embodiments, the method of treating transient neonatal myasthenia, juvenile myasthenia gravis, congenital myasthenia gravis syndromes (CMS), or any combination thereof, in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 2 weeks. In certain embodiments, the method of treating transient neonatal myasthenia, juvenile myasthenia gravis, congenital myasthenia gravis syndromes (CMS), or any combination thereof, in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 2 weeks. In certain embodiments, the method of treating transient neonatal myasthenia, juvenile myasthenia gravis, congenital myasthenia gravis syndromes (CMS), or any combination thereof, in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 2 weeks. In certain embodiments, the method of treating transient neonatal myasthenia, juvenile myasthenia gravis, congenital myasthenia gravis syndromes (CMS), or any combination thereof, in a pediatric subject in need thereof comprises administering nipocalimab intravenously to the pediatric subject, wherein the nipocalimab is administered as a loading dose at 5-60 mg/kg followed by a maintenance dose at 5-60 mg/kg every 4 weeks. In certain embodiments, the method of treating transient neonatal myasthenia, juvenile myasthenia gravis, congenital myasthenia gravis syndromes (CMS), or any combination thereof, in a pediatric subject in need thereof comprises administering nipocalimab intravenously to the pediatric subject, wherein the nipocalimab is administered as a loading dose at 5-30 mg/kg followed by a maintenance dose at 5-30 mg/kg every 4 weeks. In certain embodiments, the method of treating transient neonatal myasthenia, juvenile myasthenia gravis, congenital myasthenia gravis syndromes (CMS), or any combination thereof, in a pediatric subject in need thereof comprises administering nipocalimab intravenously to the pediatric subject, wherein the nipocalimab is administered as a loading dose at 15-30 mg/kg followed by a maintenance dose at 15-30 mg/kg every 4 weeks. In certain embodiments, the method of treating transient neonatal myasthenia, juvenile myasthenia gravis, congenital myasthenia gravis syndromes (CMS), or any combination thereof, in a pediatric subject in need thereof comprises administering a therapeutically effective amount of nipocalimab intravenously to the pediatric subject, wherein the therapeutically effective amount of nipocalimab is 30 mg/kg. In certain embodiments, the method of treating transient neonatal myasthenia, juvenile myasthenia gravis, congenital myasthenia gravis syndromes (CMS), or any combination thereof, in a pediatric subject in need thereof comprises administering a therapeutically effective amount of nipocalimab intravenously to the pediatric subject, wherein the therapeutically effective amount of nipocalimab is 15 mg/kg. In certain embodiments, the method of treating transient neonatal myasthenia, juvenile myasthenia gravis, congenital myasthenia gravis syndromes (CMS), or any combination thereof, in a pediatric subject in need thereof comprises administering nipocalimab intravenously to the pediatric subject, wherein the nipocalimab is administered as a loading dose at 30 mg/kg followed by a maintenance dose at 15 mg/kg every 2 weeks. In certain embodiments, the method of treating transient neonatal myasthenia, juvenile myasthenia gravis, congenital myasthenia gravis syndromes (CMS), or any combination thereof, in a pediatric subject in need thereof comprises administering nipocalimab intravenously to the pediatric subject, wherein the nipocalimab is administered as a loading dose at 30 mg/kg followed by a maintenance dose at 30 mg/kg every 4 weeks.

In some embodiments, the pharmaceutical composition comprises one or more of sodium phosphate, sodium chloride, trehalose, or polysorbate.

As used herein, the term “pharmaceutical composition” refers to a medicinal or pharmaceutical formulation that contains an active ingredient as well as one or more excipients and diluents to enable the active ingredient suitable for the method of administration. The pharmaceutical composition of the present disclosure includes pharmaceutically acceptable components that are compatible with the anti-FcRn antibody. The pharmaceutical composition may be in aqueous form for intravenous or subcutaneous administration or in tablet or capsule form for oral administration. In some embodiments, the composition is suitable for intravenous administration. In some embodiments, the composition is suitable for subcutaneous administration.

In some embodiments, the pharmaceutical compositions of the invention that contain an anti-FcRn antibody as the therapeutic proteins may be formulated for intravenous administration, parenteral administration, subcutaneous administration, intramuscular administration, intra-arterial administration, intrathecal administration, or intraperitoneal administration. In some embodiments, the pharmaceutical composition may also be formulated for, or administered via, oral, nasal, spray, aerosol, rectal, or vaginal administration. For injectable formulations, various effective pharmaceutical carriers are known in the art.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present disclosure, the pharmaceutically acceptable carrier must provide adequate pharmaceutical stability to the Fc construct. The nature of the carrier differs with the mode of administration. For example, for intravenous administration, an aqueous solution carrier is generally used; for oral administration, a solid carrier is preferred.

As used herein, the term “therapeutically effective amount” refers to an amount, e.g., pharmaceutical dose, effective in inducing a desired biological effect in a pediatric subject or patient or in treating a patient having a condition or disorder described herein. It is also to be understood herein that a “therapeutically effective amount” may be interpreted as an amount giving a desired therapeutic effect, either taken in one dose or in any dosage or route, taken alone or in combination with other therapeutic agents.

As used herein, the term “no more than” refers to an amount that is less than equal to. This may be an amount in integers. For example, no more than two substitutions can refer to 0, 1, or 2 substitutions.

As used herein, the terms “treatment” or “treating” refer to reducing, decreasing, decreasing the risk of, or decreasing the side effects of a particular disease or condition. Reducing, decreasing, decreasing the risk of, or decreasing the side effects of are relative to a pediatric subject who did not receive treatment, e.g., a control, a baseline, or a known control level or measurement.

Accordingly, in some embodiments, methods of treating myasthenia gravis, transient neonatal myasthenia, juvenile myasthenia gravis, congenital myasthenia gravis syndromes (CMS), or any combination thereof, in a pediatric subject are provided. In some embodiments, the myasthenia gravis is generalized myasthenia gravis. In some embodiments, the myasthenia gravis is transient neonatal myasthenia, juvenile myasthenia gravis, congenital myasthenia gravis syndromes (CMS), or any combination thereof. In some embodiments, the myasthenia gravis is transient myasthenia gravis. In some embodiments, the myasthenia gravis is juvenile myasthenia gravis. In some embodiments, the myasthenia gravis is congenital myasthenia gravis syndromes (CMS). In some embodiments, the pediatric subject is a pediatric subject with a suboptimal response to a stable therapy for gMG, transient neonatal myasthenia, juvenile myasthenia gravis, congenital myasthenia gravis syndromes (CMS), or any combination thereof. In some embodiments, the stable therapy for gMG, transient neonatal myasthenia, juvenile myasthenia gravis, congenital myasthenia gravis syndromes (CMS), or any combination thereof, comprises: acetylcholinesterase inhibitors, glucocorticosteroids, and immunosuppressants. In some embodiments, the immunosuppressants are selected from: azathioprine, mycophenolate mofetil/mycophenolic acid, methotrexate, cyclosporine, tacrolimus, and cyclophosphamide. In some embodiments, the method comprise administering a pharmaceutical composition comprising administering an anti-FcRn antibody to the pediatric subject. In some embodiments, the anti-FcRn antibody is nipocalimab, RVT-1401 (HL161), rozanolixizumab (UCB7665), ALXN1830, ABY-039, or efgartigimod. In some embodiments, the antibody is nipocalimab. In some embodiments, the anti-FcRn antibody is as provided for herein.

In some embodiments, the pediatric subject treated for myasthenia gravis shows an improvement in one or more of the following assays, scores, or criteria, which can be used to evaluate the improvement or condition of a pediatric subject with myasthenia gravis. In some embodiments, the myasthenia gravis is transient neonatal myasthenia, juvenile myasthenia gravis, congenital myasthenia gravis syndromes (CMS), or any combination thereof. In some embodiments, the myasthenia gravis is transient myasthenia gravis. In some embodiments, the myasthenia gravis is juvenile myasthenia gravis. In some embodiments, the myasthenia gravis is congenital myasthenia gravis syndromes (CMS). In some embodiments, the pediatric subject shows improvement in one or more of the following: Myasthenia Gravis—Activities of Daily Living (MG-ADL) score, Quantitative Myasthenia Gravis (QMG) score, European Quality of Life 5-Dimension Youth (EQ-5D-Y) tool, Myasthenia Gravis Quality of Life (MG-QoL 15r) score, Neurological Quality of Life (Neuro-QoL) fatigue score, Pediatric Quality of Life Inventory (PedsQL), EQ-5D-5L score, EQ-5D-5Y score, Myasthenia Gravis Foundation of America (MGFA) scale, patient global impression of severity (PGI-S) score, and patient global impression of change (PGI-C) score.

In some embodiments, the pediatric subject being treated for myasthenia gravis, or moderate to severe active myasthenia gravis, or transient neonatal myasthenia, juvenile myasthenia gravis, congenital myasthenia gravis syndromes (CMS), or any combination thereof, has or shows a reduction in one or more immunoglobulin isotypes or total IgG. In some embodiments, the subject being treated for myasthenia gravis has or shows a reduction in one or more immunoglobulin isotypes or total IgG. In some embodiments, the subject being treated for myasthenia gravis, or moderate to severe active myasthenia gravis has or shows a reduction in one or more immunoglobulin isotypes or total IgG. In some embodiments, the subject being treated for juvenile myasthenia gravis has or shows a reduction in one or more immunoglobulin isotypes or total IgG. In some embodiments, the subject being treated for transient neonatal myasthenia has or shows a reduction in one or more immunoglobulin isotypes or total IgG. In some embodiments, the subject being treated for congenital myasthenia gravis syndromes (CMS) has or shows a reduction in one or more immunoglobulin isotypes or total IgG. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, or 90%. In some embodiments, the administration of the anti-FcRn antibody reduces serum IgG by at least 10%, at least 20%, at least 30%, at least 40%, at least 50%, at least 60%, at least 70%, at least 80%, or at least 90% of baseline serum IgG. In some embodiments, the administration of the anti-FcRn antibody reduced serum IgG in the patient by at least 90% of baseline. In some embodiments, the administration of the anti-FcRn antibody reduces serum IgG in the patient by at least 80% of baseline. In some embodiments, the administration of the anti-FcRn antibody reduces serum IgG in the patient by at least 70% of baseline. In some embodiments, the isotype of immunoglobulins reduced is IgG1, IgG2, IgG3, IgG4, or any combination thereof. In some embodiments, the administration of the anti-FcRn antibody reduces serum IgG1 by at least 10%, at least 20%, at least 30%, at least 40%, at least 50%, at least 60%, at least 70%, at least 80%, or at least 90% of baseline serum IgG1. In some embodiments, the administration of the anti-FcRn antibody reduced serum IgG1 in the patient by at least 90% of baseline. In some embodiments, the administration of the anti-FcRn antibody reduces serum IgG1 in the patient by at least 80% of baseline. In some embodiments, the administration of the anti-FcRn antibody reduces serum IgG1 in the patient by at least 70% of baseline. In some embodiments, the administration of the anti-FcRn antibody reduces serum IgG2 by at least 10%, at least 20%, at least 30%, at least 40%, at least 50%, at least 60%, at least 70%, at least 80%, or at least 90% of baseline serum IgG2. In some embodiments, the administration of the anti-FcRn antibody reduced serum IgG2 in the patient by at least 90% of baseline. In some embodiments, the administration of the anti-FcRn antibody reduces serum IgG2 in the patient by at least 80% of baseline. In some embodiments, the administration of the anti-FcRn antibody reduces serum IgG2 in the patient by at least 70% of baseline. In some embodiments, the administration of the anti-FcRn antibody reduces serum IgG3 by at least 10%, at least 20%, at least 30%, at least 40%, at least 50%, at least 60%, at least 70%, at least 80%, or at least 90% of baseline serum IgG3. In some embodiments, the administration of the anti-FcRn antibody reduces serum IgG3 in the patient by at least 90% of baseline. In some embodiments, the administration of the anti-FcRn antibody reduces serum IgG3 in the patient by at least 80% of baseline. In some embodiments, the administration of the anti-FcRn antibody reduces serum IgG3 in the patient by at least 70% of baseline. In some embodiments, the administration of the anti-FcRn antibody reduces serum IgG4 by at least 10%, at least 20%, at least 30%, at least 40%, at least 50%, at least 60%, at least 70%, at least 80%, or at least 90% of baseline serum IgG4. In some embodiments, the administration of the anti-FcRn antibody reduced serum IgG4 in the patient by at least 90% of baseline. In some embodiments, the administration of the anti-FcRn antibody reduces serum IgG4 in the patient by at least 80% of baseline. In some embodiments, the administration of the anti-FcRn antibody reduces serum IgG4 in the patient by at least 70% of baseline.

In some embodiments, the pediatric subject being treated for myasthenia gravis has or shows a reduction in autoantibodies. In some embodiments, the subject being treated for myasthenia gravis has or shows a reduction in autoantibodies. In some embodiments, the subject being treated for transient neonatal myasthenia has or shows a reduction in autoantibodies. In some embodiments, the subject being treated for juvenile myasthenia gravis has or shows a reduction in autoantibodies. In some embodiments, the subject being treated for congenital myasthenia gravis syndromes (CMS) has or shows a reduction in autoantibodies. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%. In some embodiments, the autoantibodies reduced are selected from the group consisting of: anti-acetylcholine receptors (AChRs), anti-muscle-specific kinase (MuSK) anti-low-density lipoprotein receptor-related protein 4 (LRP4), anti-agrin, anti-titin, anti-Kv1.4, anti-ryanodine receptors, anti-collagen Q, and anti-cortactin. In some embodiments, the autoantibodies are anti-AChR or an anti-MuSK antibodies. In some embodiments, the administration of the anti-FcRn antibody reduces anti-AChR antibodies by at least 95%, at least 90%, at least 85%, at least 80%, at least 75%, at least 50%, or at least 25% of baseline anti-AChR antibodies. In some embodiments, the administration of the anti-FcRn antibody reduces anti-AChR antibodies by at least 95% of baseline anti-AChR antibodies. In some embodiments, the administration of the anti-FcRn antibody reduces anti-AChR antibodies by at least 90% of baseline anti-AChR antibodies. In some embodiments, the administration of the anti-FcRn antibody reduces anti-AChR antibodies by at least 85% of baseline anti-AChR antibodies. In some embodiments, the administration of the anti-FcRn antibody reduces anti-AChR antibodies by at least 80% of baseline anti-AChR antibodies. In some embodiments, the administration of the anti-FcRn antibody reduces anti-AChR antibodies by at least 75% of baseline anti-AChR antibodies. In some embodiments, the administration of the anti-FcRn antibody reduces anti-AChR antibodies by at least 50% of baseline anti-AChR antibodies. In some embodiments, the administration of the anti-FcRn antibody reduces anti-AChR antibodies by at least 25% of baseline anti-AChR antibodies. In some embodiments, the administration of the anti-FcRn antibody reduces anti-MuSK antibodies by at least 95%, at least 90%, at least 85%, at least 80%, at least 75%, at least 50%, or at least 25% of baseline anti-MuSK antibodies. In some embodiments, the administration of the anti-FcRn antibody reduces anti-MuSK antibodies by at least 95% of baseline anti-MuSK antibodies. In some embodiments, the administration of the anti-FcRn antibody reduces anti-MuSK antibodies by at least 90% of baseline anti-MuSK antibodies. In some embodiments, the administration of the anti-FcRn antibody reduces anti-MuSK antibodies by at least 85% of baseline anti-MuSK antibodies. In some embodiments, the administration of the anti-FcRn antibody reduces anti-MuSK antibodies by at least 80% of baseline anti-MuSK antibodies. In some embodiments, the administration of the anti-FcRn antibody reduces anti-MuSK antibodies by at least 75% of baseline anti-MuSK antibodies. In some embodiments, the administration of the anti-FcRn antibody reduces anti-MuSK antibodies by at least 50% of baseline anti-MuSK antibodies. In some embodiments, the administration of the anti-FcRn antibody reduces anti-MuSK antibodies by at least 25% of baseline anti-MuSK antibodies. In some embodiments, the administration of the anti-FcRn antibody reduces anti-AChR antibodies by at least 95%, at least 90%, at least 85%, at least 80%, at least 75%, at least 50%, or at least 25% of baseline anti-AChR antibodies; and anti-MuSK antibodies by at least 95%, at least 90%, at least 85%, at least 80%, at least 75%, at least 50%, or at least 25% of baseline anti-MuSK antibodies.

In some embodiments, the administration of the anti-FcRn antibody reduces serum albumin by at most 18%, at most 16%, at most 14%, at most 12%, at most 10%, at most 8%, at most 6%, at most 4%, or at most 2% of baseline of serum albumin. In some embodiments, the administration of the anti-FcRn antibody reduces serum albumin by at most 18% of baseline of serum albumin. In some embodiments, the administration of the anti-FcRn antibody reduces serum albumin by at most 16% of baseline of serum albumin. In some embodiments, the administration of the anti-FcRn antibody reduces serum albumin by at most 14% of baseline of serum albumin. In some embodiments, the administration of the anti-FcRn antibody reduces serum albumin by at most 12% of baseline of serum albumin. In some embodiments, the administration of the anti-FcRn antibody reduces serum albumin by at most 10% of baseline of serum albumin. In some embodiments, the administration of the anti-FcRn antibody reduces serum albumin by at most 8% of baseline of serum albumin. In some embodiments, the administration of the anti-FcRn antibody reduces serum albumin by at most 6% of baseline of serum albumin. In some embodiments, the administration of the anti-FcRn antibody reduces serum albumin by at most 4% of baseline of serum albumin. In some embodiments, the administration of the anti-FcRn antibody reduces serum albumin by at most 2% of baseline of serum albumin.

In some embodiments, the pediatric subject is also treated with an additional therapeutic in addition to M281. In some embodiments, the additional therapeutic is a acetylcholinesterase inhibitor, pyridostigmine, pyridostigmine bromide (Mestinon), neostigmine, prednisone, azathioprine (Imuran), mycophenylate mofetil (CellCept), tacrolimus (Prograf), methotrexate, cyclosporine (Sandimmune, Neoral), and cyclophosphamide (Cytoxan, Neosar), rituximab (Rituxan), eculizumab (Soliris), IVIg, or any combination thereof. In some embodiments, the additional therapeutic is administered concurrently or sequentially (prior to or after) with M281.

In some embodiments, the method comprise administering a pharmaceutical composition comprising administering an anti-FcRn antibody to the pediatric subject. In some embodiments, the anti-FcRn antibody is nipocalimab, RVT-1401 (HL161), rozanolixizumab (UCB7665), ALXN1830, ABY-039, or efgartigimod. In some embodiments, the antibody is nipocalimab. In some embodiments, the anti-FcRn antibody is as provided for herein. In some embodiments, the pediatric subject has or is suspected of having myasthenia gravis. In preferred embodiments, the anti-FcRn antibody is nipocalimab.

In some embodiments, administration of the anti-FcRn antibody to a pediatric subject with myasthenia gravis treats or ameliorates ocular myasthenia, ptosis, difficulty chewing, dysphagia, dysarthria, hypophonia, dyspnea, an inability to hold the mouth closed, an appearance of sadness or sleepiness, difficulty holding the head upright, diplopia, dysarthria, difficulty swallowing, change in facial expression, shortness of breath, weakness in arms, weakness in hands, weakness in fingers, weakness in legs, weakness in neck.

In some embodiments, treatment of myasthenia gravis, transient neonatal myasthenia, juvenile myasthenia gravis, congenital myasthenia gravis syndromes (CMS), or any combination thereof, includes the improvement of a clinical marker for MG progression. These markers include MG activity of daily living profile (MG-ADL), and quantitative Myasthenia Gravis (QMG) score for disease severity. In certain embodiments, MG-ADL is the primary objective for measuring improvement of MG.

The MG-ADL is an 8-point questionnaire that focuses on relevant symptoms and functional performance of activities of daily living (ADL) in MG pediatric subjects. The 8 items of the MG-ADL are derived from symptom-based components of the original 13-item QMG to assess disability secondary to ocular (2 items), bulbar (3 items), respiratory (1 item), and gross motor or limb (2 items) impairment related to effects from MG. In this functional status instrument, each response is graded 0 (normal) to 3 (most severe). The range of total MG-ADL score is 0-24. A clinically meaningful improvement in a patient's MG-ADL would be a 2 point or greater reduction in score after 2, 4, 5, 6, 7, 8, 9, 10, 11, 12, 13, 14, 15, 16, 17, 18, 19, 20, 21, 22, and 24 weeks of treatment.

In some embodiments, the pediatric patient being treated by the methods provided herein experiences a change from baseline in Myasthenia Gravis Activities of Daily Living (MG-ADL) score over time or after 2, 4, 5, 6, 7, 8, 9, 10, 11, 12, 13, 14, 15, 16, 17, 18, 19, 20, 21, 22, and 24 weeks of treatment. In some embodiments, the change from baseline of MG-ADL score is greater than or equal to 2 points on the MG-ADL scale.

The current QMG scoring system consists of 13 items: ocular (2 items), facial (1 item), bulbar (2 items), gross motor (6 items), axial (1 item), and respiratory (1 item); each graded 0 to 3, with 3 being the most severe. The range of total QMG score is 0-39. The QMG scoring system is considered to be an objective evaluation of therapy for MG and is based on quantitative testing of sentinel muscle groups. Higher scores indicated greater weakness. The QMG is preferably administered by a trained qualified healthcare professional (e.g., physician, physician assistant, nurse practitioner, nurse). The QMG is preferably to be administered by the same healthcare professional for a given pediatric subject throughout the study, if possible, and is preferably to be performed at approximately the same time of day throughout the study.

In some embodiments, the pediatric patient being treated by the methods provided herein experiences a change from baseline in QMG score after 22, 23, and 24 weeks of treatment. In some embodiments, the pediatric patient being treated by the methods provided herein experiences a change from baseline in QMG score over time after administration of the last dose. In some embodiments, the change from baseline is at least a 2, 3, 4, 5, 6, 7, or greater than or equal to 8 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose. In a some embodiments, the change from baseline is at least a 2 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change from baseline is at least a 3 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose. In a some embodiments, the change from baseline is at least a 4 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change from baseline is at least a 5 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change from baseline is at least a 6 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose. In a some embodiments, the change from baseline is at least a 7 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change from baseline is greater than or equal to 8 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose.

The 15-item Myasthenia Gravis Qualify of Life scale (MG-QoL 15) is a health-related quality of life evaluative instrument specific to subjects with MG. MG-QoL15 is preferably designed to provide information about subjects' perception of impairment and disability and the degree to which disease manifestations are tolerated and to be easy to administer and interpret. The MG-QoL 15 is completed by the subject. Total scores range from 0 to 60 and higher scores indicate greater extent of and dissatisfaction with MG-related dysfunction. The MG-QoL 15 is preferably used to assess the subject's limitations related to living with MG. Each of the 15 items were rated by the subject on a 3-point scale based on a recall period of “over the past few weeks”, with a maximum score of 30. Higher scores indicated more limitation.

In some embodiments, the pediatric patient being treated by the methods provided herein experiences a change from baseline in MG-QoL15 score after 2, 4, 5, 6, 7, 8, 9, 10, 11, 12, 13, 14, 15, 16, 17, 18, 19, 20, 21, 22, and 24 weeks of treatment. In some embodiments, the pediatric patient being treated by the methods provided herein experiences a change from baseline in MG-QoL 15 score over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

The Myasthenia Gravis Foundation of America Clinical Classification (MGFA) can be used to assess the subject's MG severity. The system comprises 5 classes of disease severity ranging from Class I (ocular muscle weakness only) to Class V (the subject is intubated). Classes II through IV are each further divided into 2 subclasses based on which muscle groups are primarily affected. The MGFA is preferably administered by a trained qualified healthcare professional (e.g., physician, physician assistant, nurse practitioner, nurse and is preferably to be assessed by the same person for a given subject throughout the study, if possible.

In some embodiments, the pediatric patient being treated by the methods provided herein experiences a shift in MGFA classification after 2, 4, 5, 6, 7, 8, 9, 10, 11, 12, 13, 14, 15, 16, 17, 18, 19, 20, 21, 22, and 24 weeks of treatment. In some embodiments, the patient being treated by the methods provided herein experiences a shift in MGFA classification over time after administration of the last dose. In some embodiments, the shift is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, or 80%.

In some embodiments, the pediatric patient being treated by the methods provided herein experiences a change on the Quality of Life in Neurological Disorders (Neuro-QoL-Fatigue). Neuro-QoL-Fatigue is a reliable and effective short 19-item fatigue survey that is filled out by the subject on all items. Higher scores indicate greater impact of MG on heavier fatigue and activity. The clinically significant improvement in the patient's Neuro-QoL-Fatigue score is reflected in the decrease in score after treatment or over time after administration of the last dose.

In some embodiments, the pediatric patient being treated by the methods provided herein experiences a change from baseline on the Neuro-QoL-Fatigue scale after 2, 4, 5, 6, 7, 8, 9, 10, 11, 12, 13, 14, 15, 16, 17, 18, 19, 20, 21, 22, and 24 weeks of treatment. In some embodiments, the patient being treated by the methods provided herein experiences a change from baseline on the Neuro-QoL-Fatigue scale over time after administration of the last dose. In some embodiments, the change from baseline on the Neuro-QoL-Fatigue indicates improvement. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the pediatric patient being treated by the methods provided herein experiences a change on the EuroQol 5-dimension 5-level quality of life questionnaire (5Q-5D-5L). The EQ-5D-5L is a standardized measure of health status developed by the EuroQol Group to provide a simple, generic measure of health for clinical and economic appraisal. The EQ-5D-5L, as a measure of health-related quality of life, defines health in terms of 5 dimensions: mobility, self-care, usual activities, pain/discomfort, anxiety/depression. Each dimension has 3 ordinal levels of severity: “no problem” (1), “some problems” (2), “severe problems” (3). Overall health state is defined as a 5-digit number. Health states defined by the 5-dimensional classification can be converted into corresponding index scores that quantify health status, where −0.594 represents “severe problems” and 1 represents “no problem.”

In some embodiments, the pediatric patient being treated by the methods provided herein experiences a change from baseline on the EQ-5D-5L scale. In some embodiments, the change from baseline on the EQ-5D-5L scale indicates improvement. In some embodiments, the patient being treated by the methods provided herein experiences a change from baseline on EQ-5D-5L scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the pediatric patient being treated by the methods provided herein experiences a change from baseline on the EQ-5D-5Y scale. The EQ-5D-Y is a standardized child friendly instrument for use as a measure of health status, primarily designed for self-completion by children and adolescents, or via a proxy version to be completed by the child's caregiver. The EQ-5D-Y descriptive system comprises the following 5 dimensions: mobility; looking after myself (washing and dressing); usual activities; pain or discomfort; and feeling worried or unhappy. Each of the 5 dimensions is divided into 3 levels of perceived problems (Level 1 indicating no problem, Level 2 indicating some problems, Level 3 a lot of problems) (EuroQol 2021; EuroQol 2019). The participant selects an answer for each of the 5 dimensions considering the response that best matches his or her health “today.” The descriptive system can be represented as a health state. The time taken to complete the questionnaire varies with age, health status, and setting but is likely to be approximately 1 minute.

In some embodiments, the pediatric patient being treated by the methods provided herein experiences a change from baseline on the EQ-5D-5Y scale. In some embodiments, the change from baseline on the EQ-5D-5Y scale indicates improvement. In some embodiments, the patient being treated by the methods provided herein experiences a change from baseline on EQ-5D-5Y scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the pediatric patient being treated by the methods provided herein experiences a change on the Patient Global Impression of Change scale (PGI-C). The PGI-C is a patient-rated assessment of response to treatment on a 7-point Likert scale.

In some embodiments, the pediatric patient being treated by the methods provided herein experiences a change from baseline on the PGI-C scale. In some embodiments, the change from baseline on the PGI-C scale indicates improvement. In some embodiments, the pediatric patient being treated by the methods provided herein experiences a change from baseline on PGI-C scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the pediatric patient being treated by the methods provided herein experiences a change on the Patient Global Impression of Severity scale (PGI-S). The Patient Global Impression of Severity (PGI-S) is a global index that may be used to rate the severity of a specific condition (a single-state scale). It is a simple, direct, easy to use scale that is intuitively understandable to clinicians. The PGI-S is a single question asking the patient to rate how their urinary tract condition is now on a scale of 1 (normal) to 4 (severe).

In some embodiments, the pediatric patient being treated by the methods provided herein experiences a change from baseline on the PGI-S scale. In some embodiments, the change from baseline on the PGI-S scale indicates improvement. In some embodiments, the pediatric patient being treated by the methods provided herein experiences a change from baseline on PGI-S scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the patient being treated by the methods provided herein experiences a change on the Columbia—Suicide Severity Rating Scale (C-SSRS). The C-SSRS is used to rate the patient's degree of suicidal ideation on a scale ranging from “no suicidal ideation” to “active suicidal ideation with specific plan and intent”. (Posner 2011)

In some embodiments, the pediatric patient being treated by the methods provided herein experiences a change from baseline on C-SSRS scale. In some embodiments, the change from baseline on C-SSRS scale indicates improvement. In some embodiments, the pediatric patient being treated by the methods provided herein experiences a change from baseline on C-SSRS scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the patient being treated by the methods provided herein experiences a change on the Pediatric Quality of Life Inventory scale (PedsQL). The PedsQL Measurement Model is a modular approach to measuring health-related quality of life (HRQOL) in healthy children and adolescents and those with acute and chronic health conditions. The PedsQL Measurement Model integrates seamlessly both generic core scales and disease-specific modules into one measurement system. The 23-item PedsQL Generic Core Scales were designed to measure the core dimensions of health as delineated by the World Health Organization, as well as role (school) functioning. The PedsQL can be completed by children and young people, with versions available for children and young people aged 5-7, 8-12, and 13-18. Parent-rated versions are available for children aged 2-4, 5-7, 8-12, and 13-18. The PedsQL inventory takes around five minutes to complete and can be self-administered by parents, children and young people aged 8 to 18 after being introduced by a trained administrator. For younger children and as an alternative in special circumstances, clinicians can administer the inventory as long as instructions and all items are read word-for-word to the child or young person. Items on the PedsQL Generic Core Scales are reverse scored and transformed to a 0-100 scale. Higher scores indicate better health related quality of life: 0 (“Never”)=100; 1 (“Almost Never”)=75; 2 (“Sometimes”)=50; 3 (“Often”)=25; and 4 (“Almost Always”)=0. Versions used: Teen report acute version for children ages 13-18; Parent report acute version for children ages 8-12; Parent report acute version for young children ages 5-7 and Parent report acute version for toddlers ages 2-4.

In some embodiments, the pediatric patient being treated by the methods provided herein experiences a change from baseline on PedsQL scale. In some embodiments, the change from baseline on PedsQL scale indicates improvement. In some embodiments, the pediatric patient being treated by the methods provided herein experiences a change from baseline on PedsQL scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

As provided for herein, in some embodiments, pharmaceutical compositions comprising an anti-FcRn antibody are provided. In some embodiments, pharmaceutical compositions comprising an anti-FcRn antibody for administration to a pediatric patient suffering from myasthenia gravis wherein the anti-FcRn antibody is administered to the patient in a therapeutically effective amount from about 1 mg/kg to about 100 mg/kg, or about 5 mg/kg to about 60 mg/kg every 2 weeks are provided. In some embodiments, the myasthenia gravis is generalized myasthenia gravis. In some embodiments, the myasthenia gravis is transient neonatal myasthenia, juvenile myasthenia gravis, congenital myasthenia gravis syndromes (CMS), or any combination thereof. In some embodiments, the myasthenia gravis is transient myasthenia gravis. In some embodiments, the myasthenia gravis is juvenile myasthenia gravis. In some embodiments, the myasthenia gravis is congenital myasthenia gravis syndromes (CMS). In some embodiments, the pediatric subject is a subject with a suboptimal response to a stable therapy for gMG. In some embodiments, the stable therapy for gMG comprises: acetylcholinesterase inhibitors, glucocorticosteroids, and immunosuppressants. In some embodiments, the immunosuppressants are selected from: azathioprine, mycophenolate mofetil/ mycophenolic acid, methotrexate, cyclosporine, tacrolimus, and cyclophosphamide. In some embodiments, the method comprise administering a pharmaceutical composition comprising administering an anti-FcRn antibody to the pediatric subject. In some embodiments, the anti-FcRn antibody is nipocalimab, RVT-1401 (HL161), rozanolixizumab (UCB7665), ALXN1830, ABY-039, or efgartigimod. In some embodiments, the anti-FcRn antibody is nipocalimab.

In some embodiments, the pharmaceutical composition comprises a therapeutically effective amount of the anti-FcRn antibody. In some embodiments, the pharmaceutical composition comprises a therapeutically effective amount of the anti-FcRn antibody. In preferred embodiments, the anti-FcRn antibody is preferably nipocalimab. In some embodiments, the therapeutically effective amount is from about 1 mg/kg to about 100 mg/kg, about 5 mg/kg to about 60 mg/kg, about 5 mg/kg to about 15 mg/kg, about 15 mg/kg to about 60 mg/kg, about 15 mg/kg to about 30 mg/kg, or about 30 mg/kg to about 60 mg/kg of the anti-FcRn antibody. In some embodiments, the therapeutically effective amount is about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, or about 60 mg/kg. In some embodiments, the therapeutically effective amount is 30 mg/kg. In some embodiments, the therapeutically effective amount is 15 mg/kg.

In some embodiments, the pharmaceutical composition is administered every week, every two weeks, or monthly. In some embodiments, the pharmaceutical composition is administered to the subject as an initial loading dose and the maintenance dose, and comprises: an initial loading dose comprising the anti-FcRn antibody at a dose of about 1 mg/kg to about 100 mg/kg, about 5 mg/kg to about 60 mg/kg, about 5 mg/kg to about 15 mg/kg, about 15 mg/kg to about 60 mg/kg, about 15 mg/kg to about 30 mg/kg, or about 30 mg/kg to about 60 mg/kg; and administered to the subject a maintenance dose comprising the anti-FcRn antibody at a dose of about 5 mg/kg to about 45 mg/kg, about 5 mg/kg to about 15 mg/kg, about 15 mg/kg to about 45 mg/kg, or about 30 mg/kg to about 45 mg/kg. In some embodiments, the pharmaceutical composition is administered to the subject as an initial loading dose and the maintenance dose, and comprises: an initial loading dose comprising the anti-FcRn antibody at a dose of 30 mg/kg; and administered to the subject a maintenance dose comprising the anti-FcRn antibody at a dose of 15 mg/kg. In preferred embodiments, the anti-FcRn antibody is preferably nipocalimab.

In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in the patient showing improvement in MG-ADL score, QMG score, MG-QoL 15r score, Neuro-QoL-Fatigue score, EQ-5D-5L score, EQ-5D-5Y score, MGFA scale, PGI-C score, PGI-S score, and PedsQL score.

In some embodiments, the administration of the pharmaceutical composition to the pediatric patient to treat myasthenia gravis treats, transient neonatal myasthenia, juvenile myasthenia gravis, congenital myasthenia gravis syndromes (CMS), or any combination thereof, or ameliorates ocular myasthenia, ptosis, difficulty chewing, dysphagia, dysarthria, hypophonia, dyspnea, an inability to hold the mouth closed, an appearance of sadness or sleepiness, difficulty holding the head upright, diplopia, dysarthria, difficulty swallowing, change in facial expression, shortness of breath, weakness in arms, weakness in hands, weakness in fingers, weakness in legs, weakness in neck.

In some embodiments, the administration of the pharmaceutical composition to the pediatric patient shows a reduction in one or more immunoglobulin isotypes or total IgG in the patient. In some embodiments, the isotype is IgG1, IgG2, IgG3, IgG4, IgA, IgM or IgE. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, or 80%. In some embodiments, the isotype of immunoglobulins reduced is IgG1, IgG2, IgG3, IgG4, IgA, IgM or IgE, or any combination thereof.

In some embodiments, the administration of the pharmaceutical composition to the pediatric patient shows a reduction in autoantibodies in the patient. In some embodiments, the autoantibodies are selected from the group consisting of: anti-acetylcholine receptors (AChRs), anti-muscle-specific kinase (MuSK) anti-low-density lipoprotein receptor-related protein 4 (LRP4), anti-agrin, anti-titin, anti-Kv1.4, anti-ryanodine receptors, anti-collagen Q, and anti-cortactin. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the pharmaceutical composition is also administered with an additional therapeutic in addition to nipocalimab. In some embodiments, the additional therapeutic is a acetylcholinesterase inhibitor, pyridostigmine, pyridostigmine bromide (Mestinon), neostigmine, prednisone, azathioprine (Imuran), mycophenylate mofetil (CellCept), tacrolimus (Prograf), methotrexate, cyclosporine (Sandimmune, Neoral), and cyclophosphamide (Cytoxan, Neosar), rituximab (Rituxan), eculizumab (Soliris), IVIg, or any combination thereof. In some embodiments, the additional therapeutic is administered concurrently or sequentially (prior to or after) with nipocalimab.

In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in the pediatric patient showing change from baseline of MG-ADL score. In some embodiments, the administration of the pharmaceutical composition to the pediatric patient to treat myasthenia gravis results in a change from baseline of MG-ADL score that is greater than or equal to 2 points on the MG-ADL scale. In some embodiments, the administration of the pharmaceutical composition to the pediatric patient results in an improvement in the patient as measured by ACR score over time or 2, 4, 5, 6, 7, 8, 9, 10, 11, 12, 13, 14, 15, 16, 17, 18, 19, 20, 21, 22, and 24 weeks after administration of the first dose of the pharmaceutical composition to the pediatric patient.

In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in the patient showing a change from baseline in QMG score after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the administration of the pharmaceutical composition to the pediatric patient to treat myasthenia gravis results in the patient showing a change from baseline in QMG score over time after administration of the last dose. In a some embodiments, the change from baseline is at least a 2, 3, 4, 5, 6, 7, or greater than or equal to 8 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose of the pharmaceutical composition. In a some embodiments, the change from baseline is at least a 2 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose of the pharmaceutical composition. In a some embodiments, the change from baseline is at least a 3 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose of the pharmaceutical composition. In a some embodiments, the change from baseline is at least a 4 point reduction in the patient's QMG score 2, 4, 5, 6, 7, 8, 9, 10, 11, 12, 13, 14, 15, 16, 17, 18, 19, 20, 21, 22, and 24 weeks of treatment or over time after administration of the last dose of the pharmaceutical composition. In a some embodiments, the change from baseline is at least a 5 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose of the pharmaceutical composition. In a some embodiments, the change from baseline is at least a 6 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose of the pharmaceutical composition. In a some embodiments, the change from baseline is at least a 7 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose of the pharmaceutical composition. In a some embodiments, the change from baseline is greater than or equal to 8 point reduction in the patient's QMG score after 2, 4, 5, 6, 7, 8, 9, 10, 11, 12, 13, 14, 15, 16, 17, 18, 19, 20, 21, 22, and 24 weeks of treatment or over time after administration of the last dose of the pharmaceutical composition.

In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in the patient showing a change from baseline in MG-QoL15 score after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in the patient showing a change from baseline in MG-QoL15 score over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in the patient showing a shift in MGFA classification after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the administration of the pharmaceutical composition to the pediatric patient to treat myasthenia gravis results in the patient showing a shift in MGFA classification over time after administration of the last dose of the pharmaceutical composition. In some embodiments, the shift is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, or 80%.

In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in a change from baseline on the Neuro-QoL-Fatigue scale after 2, 4, 5, 6, 7, 8, 9, 10, 11, 12, 13, 14, 15, 16, 17, 18, 19, 20, 21, 22, and 24 weeks of treatment. In some embodiments, the administration of the pharmaceutical composition to the pediatric patient to treat myasthenia gravis results in a change from baseline on the Neuro-QoL-Fatigue scale over time after administration of the last dose. In some embodiments, the change from baseline on the Neuro-QoL-Fatigue indicates improvement. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the administration of the pharmaceutical composition to the pediatric patient to treat myasthenia gravis results, transient neonatal myasthenia, juvenile myasthenia gravis, congenital myasthenia gravis syndromes (CMS), or any combination thereof, in a change from baseline on the EQ-5D-5L scale. In some embodiments, the change from baseline on the EQ-5D-5L scale indicates improvement. In some embodiments, the administration of the pharmaceutical composition to the pediatric patient to treat myasthenia gravis results in a change from baseline on EQ-5D-5L scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the administration of the pharmaceutical composition to the pediatric patient to treat myasthenia gravis results, transient neonatal myasthenia, juvenile myasthenia gravis, congenital myasthenia gravis syndromes (CMS), or any combination thereof, in a change from baseline on the EQ-5D-5Y scale. In some embodiments, the change from baseline on the EQ-5D-5Y scale indicates improvement. In some embodiments, the administration of the pharmaceutical composition to the pediatric patient to treat myasthenia gravis results in a change from baseline on EQ-5D-5Y scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in a change from baseline on the PGI-C scale. In some embodiments, the change from baseline on the PGI-C scale indicates improvement. In some embodiments, the administration of the pharmaceutical composition to the pediatric patient to treat myasthenia gravis results in a change from baseline on PGI-C scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in a change from baseline on the PGI-S scale. In some embodiments, the change from baseline on the PGI-S scale indicates improvement. In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in a change from baseline on PGI-S scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments the administration of the pharmaceutical composition to the pediatric patient to treat myasthenia gravis, transient neonatal myasthenia, juvenile myasthenia gravis, congenital myasthenia gravis syndromes (CMS), or any combination thereof, results in a change from baseline on the C-SSRS scale. In some embodiments, the change from baseline on the C-SSRS scale indicates improvement. In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in a change from baseline on C-SSRS scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in a change from baseline on PedsQL scale. In some embodiments, the change from baseline on PedsQL scale indicates improvement. In some embodiments, the administration of the pharmaceutical composition to the pediatric patient to treat myasthenia gravis, transient neonatal myasthenia, juvenile myasthenia gravis, congenital myasthenia gravis syndromes (CMS), or any combination thereof, results in a change from baseline on PedsQL scale after 2, 4, 5, 6, 7, 8, 9, 10, 11, 12, 13, 14, 15, 16, 17, 18, 19, 20, 21, 22, and 24 weeks of treatment or over time after administration of the last dose. In some embodiments, the change is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

In some embodiments, the subject being treated for myasthenia gravis, transient neonatal myasthenia, juvenile myasthenia gravis, congenital myasthenia gravis syndromes (CMS), or any combination thereof, with an anti-FcRn antibody does not experience clinically significant increased levels of total cholesterol, high-density lipoprotein (HDL), calculated low-density lipoprotein (LDL), and triglycerides after being treated with the antibody. In some embodiments, the anti-FcRn antibody is M281. In some embodiments, the subject being treated for myasthenia gravis, transient neonatal myasthenia, juvenile myasthenia gravis, congenital myasthenia gravis syndromes (CMS), or any combination thereof, with an anti-FcRn antibody does not experience clinically significant increased levels of total cholesterol after being treated with the antibody. In some embodiments, the subject being treated for myasthenia gravis, transient neonatal myasthenia, juvenile myasthenia gravis, congenital myasthenia gravis syndromes (CMS), or any combination thereof, with an anti-FcRn antibody does not experience clinically significant increased levels of high-density lipoprotein (HDL) after being treated with the antibody. In some embodiments, the subject being treated for myasthenia gravis, transient neonatal myasthenia, juvenile myasthenia gravis, congenital myasthenia gravis syndromes (CMS), or any combination thereof, with an anti-FcRn antibody does not experience clinically significant increased levels of calculated low-density lipoprotein (LDL) after being treated with the antibody. In some embodiments, the subject being treated for myasthenia gravis, transient neonatal myasthenia, juvenile myasthenia gravis, congenital myasthenia gravis syndromes (CMS), or any combination thereof, with an anti-FcRn antibody does not experience clinically significant increased levels of triglycerides after being treated with the antibody. In some embodiments, the subject being treated for myasthenia gravis, transient neonatal myasthenia, juvenile myasthenia gravis, congenital myasthenia gravis syndromes (CMS), or any combination thereof, with an anti-FcRn antibody does not experience clinically significant increased levels of total cholesterol after being treated with M281. In some embodiments, the subject being treated myasthenia gravis, transient neonatal myasthenia, juvenile myasthenia gravis, congenital myasthenia gravis syndromes (CMS), or any combination thereof, with an anti-FcRn antibody does not experience significantly increased levels of high-density lipoprotein (HDL) after being treated with M281. In some embodiments, the subject being treated for myasthenia gravis, transient neonatal myasthenia, juvenile myasthenia gravis, congenital myasthenia gravis syndromes (CMS), or any combination thereof, with an anti-FcRn antibody does not experience clinically significant increased levels of calculated low-density lipoprotein (LDL) after being treated with M281. In some embodiments, the subject being treated for myasthenia gravis, transient neonatal myasthenia, juvenile myasthenia gravis, congenital myasthenia gravis syndromes (CMS), or any combination thereof, with an anti-FcRn antibody does not experience clinically significant increased levels of triglycerides after being treated with M281.

In some embodiments, the administration of the pharmaceutical composition to the patient does not significantly increase levels of total cholesterol, high-density lipoprotein (HDL), calculated low-density lipoprotein (LDL), and triglycerides after administration of the pharmaceutical composition comprising the antibody. In some embodiments, significant increase is a clinically significant increase. In some embodiments, the anti-FcRn antibody is M281. In some embodiments, the administration of the pharmaceutical composition to the patient does not significantly increase levels of total cholesterol after administration of the pharmaceutical composition comprising the antibody. In some embodiments, the administration of the pharmaceutical composition to the patient does not significantly increase levels of high-density lipoprotein (HDL) after administration of the pharmaceutical composition comprising the antibody. In some embodiments, the administration of the pharmaceutical composition to the patient does not significantly increase levels of calculated low-density lipoprotein (LDL) after administration of the pharmaceutical composition comprising the antibody. In some embodiments, the administration of the pharmaceutical composition to the patient does not significantly increase levels of triglycerides after administration of the pharmaceutical composition comprising the antibody. In some embodiments, the administration of the pharmaceutical composition to the patient does not significantly increase levels of total cholesterol after administration of the pharmaceutical composition comprising M281. In some embodiments, the administration of the pharmaceutical composition to the patient does not significantly increase levels of high-density lipoprotein (HDL) after administration of the pharmaceutical composition comprising M281. In some embodiments, the administration of the pharmaceutical composition to the patient does not significantly increase levels of calculated low-density lipoprotein (LDL) after administration of the pharmaceutical composition comprising M281. In some embodiments, the administration of the pharmaceutical composition to the patient does not significantly increase levels of triglycerides after administration of the pharmaceutical composition comprising M281.

As used herein, the phrase “does not significantly increase” when used in reference to levels (measurements) of total cholesterol, high-density lipoprotein (HDL), calculated low-density lipoprotein (LDL), or triglycerides” means that any increase is less than 30% as compared to the level(s) prior (baseline) to the administration of the antibody or compositions provided for herein. As used herein, the phrase “does not experience clinically significant increased” when used in reference to levels (measurements) of total cholesterol, high-density lipoprotein (HDL), calculated low-density lipoprotein (LDL), or triglycerides” means that any increase is less than 30% as compared to the level(s) prior (baseline) to the administration of the antibody or compositions provided for herein. In some embodiments, the increase is less than 25%, 20%, 15%, 10%, or 5%. In some embodiments, the increase is no greater than about 1 to about 30%, about 5% to about 25%, about 1% to about 20%, about 1% to about 15%, about 1% to about 10%, about 5% to about 15%, about 5% to about 20%, about 10% to about 20%, about 1%, about 5%, about 10%, about 15%, about 20%, about 25%, or about 30%.

As used herein, the term “change” in reference to a baseline refers to a subject having an improvement as compared to that subject's conditions, scores, symptoms, and the like prior to being treated with the anti-FcRn antibodies as provided for herein.

“Baseline” refers to a subject prior to being treated with a therapeutic, such as the anti-FcRn antibodies, including those provided for herein.

As provided herein, in some embodiments, the improvement in symptoms or conditions are referred to as occurring 24 weeks after initiation of treatment. Although reference is made to determining whether those improvements are measurable at 24 weeks after initiation of treatment, in some embodiments, the improvements or changes described herein will occur within 2, 4, 6, 8, 10, 12, 14, 16, 18, 20, 22, and 24 weeks. In some embodiments, the changes or improvements will last at least 2, 4, 6, 8, 10, 12, 14, 16, 18, 20, 22, and 24 weeks.

In some embodiments, the subject being treated for myasthenia gravis is a subject in need thereof.

As used herein and in the appended claims, the singular forms “a”, “an” and “the” include plural reference unless the context clearly dictates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “individual,” “subject,” or “patient,” can be used interchangeably, means any animal, including mammals, such as mice, rats, other rodents, rabbits, dogs, cats, swine, cattle, sheep, horses, or primates, such as humans. As used herein, the term “mammal” means a rodent (i.e., a mouse, a rat, or a guinea pig), a monkey, a cat, a dog, a cow, a horse, a pig, or a human. In some embodiments, the mammal is a human. In some embodiments, the subject is a pediatric subject.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

The following examples are illustrative, but not limiting, of the compounds, compositions and methods described herein. Other suitable modifications and adaptations known to those skilled in the art are within the scope of the following embodiments.

Other Embodiments

While the disclosure has been described in connection with specific embodiments thereof, it will be understood that it is capable of further modifications and this application is intended to cover any variations, uses, or adaptations following, in general, the principles and including such departures from the present disclosure come within known or customary practice within the art to which the disclosure pertains and may be applied to the essential features hereinbefore set forth.

In some embodiments, embodiments provided herein also include, but are not limited to:
1. A method of treating pediatric myasthenia gravis in a pediatric patient in need thereof, the method comprising administering an initial loading dose of about 30 mg/kg to about 60 mg/kg of an anti-FcRn antibody followed by administering a maintenance dose of about 15 mg/kg to about 30 mg/kg of the anti-FcRn antibody, wherein the anti-FcRn antibody comprises: a heavy chain comprising a HCDR1 of SEQ ID NO: 6, a HCDR2 of SEQ ID NO: 7, and a HCDR3 of SEQ ID NO: 8; and
a light chain comprising a LCDR1 of SEQ ID NO: 3, a LCDR2 of SEQ ID NO: 4, and a LCDR3 of SEQ ID NO: 5;
wherein the administration reduces serum IgG in the patient by at least 10%, at least 20%, at least 30%, at least 40%, at least 50%, at least 60%, at least 70%, at least 80%, or at least 90% of baseline serum IgG, and
wherein the pediatric myasthenia gravis is selected from transient neonatal myasthenia, juvenile myasthenia gravis, congenital myasthenia gravis syndrome, or any combination thereof.
2. The method of embodiment 1, wherein the pediatric myasthenia gravis is transient neonatal myasthenia.
3. The method of embodiment 1, wherein the pediatric myasthenia gravis is juvenile myasthenia gravis.
4. The method of embodiment 1, wherein the pediatric myasthenia gravis is congenital myasthenia gravis syndrome.
5. The method of embodiment 1, wherein the heavy chain comprises an amino acid sequence having at least 95% identity to the sequence of SEQ ID NO: 2 and the light chain comprises an amino acid sequence having at least 95% identity to the sequence of SEQ ID NO: 1.
6. The method of embodiment 1, wherein the heavy chain comprises a variable region heavy chain comprising an amino acid sequence having at least 95% identity to the sequence of SEQ ID NO: 10 and the light chain comprises a variable region light chain comprising an amino acid sequence having at least 95% identity to the sequence of SEQ ID NO: 9.
7. The method of embodiment 1, wherein the heavy chain comprises the amino acid sequence of SEQ ID NO: 2 and the light chain comprises the amino acid sequence of SEQ ID NO: 1.
8. The method of embodiment 1, wherein the heavy chain comprises a variable region heavy chain comprising the amino acid sequence of SEQ ID NO: 10 and the light chain comprises a variable region light chain comprising the amino acid sequence of SEQ ID NO: 9.
9. The method of any one of embodiments 1-8, wherein the administration is intravenous or subcutaneous.
10. The method of any one of embodiments 1-9, wherein the administration comprises administering a pharmaceutical composition comprising about 10 mg/ml to about 60 mg/ml of the anti-FcRn antibody, about 20 mM to about 30 mM sodium phosphate, about 20 mM to about 30 mM sodium chloride, about 80 mg/ml to about 100 mg/ml Trehalose, and about 0.1% w/v to about 0.005% w/v Polysorbate 80.
11. The method of any one of embodiments 1-10, wherein the initial loading dose is about 60 mg/kg or about 30 mg/kg.
12. The method of any one of embodiment 1-11, wherein the maintenance dose is about 15 mg/kg or about 30 mg/kg.
13. The method of any one of embodiments 1-12, wherein the maintenance dose is administered:
1 week, 2 weeks, 3 weeks, 4 weeks, or monthly after the administration of the initial loading dose; and
1 week, 2 weeks, 3 weeks, 4 weeks, or monthly after the administration of the preceding maintenance dose.
14. The method of any one of embodiments 1-13, wherein:
the initial loading dose is infused into the pediatric patient in about 30 minutes to about 90 minutes; and
the maintenance dose is infused into the pediatric patient in about 15 to about 60 minutes.
15. The method of any one of embodiments 1-14, wherein the serum IgG is IgG1, IgG2, IgG3, or IgG4, or any combination thereof, and wherein the reduction is by at least 20% of baseline, or at least 30% of baseline.
16. The method of any one of embodiments 1-15, wherein the administration of the anti-FcRn antibody reduces serum albumin by at most 18%, at most 16%, at most 14%, at most 12%, at most 10%, at most 8%, at most 6%, at most 4%, or at most 2% of baseline of serum albumin.
17. The method of any one of embodiments 1-16, wherein the administration reduces serum autoantibodies, wherein:
the autoantibodies are selected from the group consisting of: anti-acetylcholine receptors (AChRs), anti-muscle-specific kinase (MuSK) anti-low-density lipoprotein receptor-related protein 4 (LRP4), anti-agrin, anti-titin, anti-Kv1.4, anti-ryanodine receptors, anti-collagen Q, and anti-cortactin; and
the reduction is by at least 95%, at least 90%, at least 85%, at least 80%, at least 75%, at least 50%, or at least 25% of baseline serum autoantibodies.
18. The method of embodiment 17, wherein the administration of the anti-FcRn antibody reduces anti-AChR antibodies by at least 95%, at least 90%, at least 85%, at least 80%, at least 75%, at least 50%, or at least 25% of baseline anti-AChR antibodies.
19. The method of any one of embodiments 17 or 18, wherein the administration of the anti-FcRn antibody reduces anti-MuSK antibodies by at least 95%, at least 90%, at least 85%, at least 80%, at least 75%, at least 50%, or at least 25% of baseline anti-MuSK antibodies.
20. The method of any one of embodiments 1-19, wherein the patient achieves a change from baseline in MG-ADL score, QMG score, Neuro-QoL-Fatigue score, EQ-5D-5Y score, PGI-C score, PGI-S score, PedsQL score, or any combination thereof.
21. The method of any one of embodiments 1-20, wherein the administration of the anti-FcRn antibody to the pediatric patient does not significantly increase levels of total cholesterol, HDL, calculated LDL, and triglycerides in the subject as compared to the levels prior to the administration of the anti-FcRn antibody.
22. A pharmaceutical composition comprising an anti-FcRn antibody for administration to a pediatric patient suffering from pediatric myasthenia gravis, wherein:
the anti-FcRn antibody is administered to the pediatric patient intravenously or subcutaneously at an initial loading dose of about 30 mg/kg to about 60 mg/kg followed by administering a maintenance dose of about 15 mg/kg to about 30 mg/kg of the anti-FcRn antibody; and the anti-FcRn antibody comprises:
a heavy chain comprising a HCDR1 of SEQ ID NO: 6, a HCDR2 of SEQ ID NO: 7, and a HCDR3 of SEQ ID NO: 8; and
a light chain comprising a LCDR1 of SEQ ID NO: 3, a LCDR2 of SEQ ID NO: 4, and a LCDR3 of SEQ ID NO: 5, and
wherein the pediatric myasthenia gravis is selected from transient neonatal myasthenia, juvenile myasthenia gravis, congenital myasthenia gravis syndromes, or any combination thereof.
23. The pharmaceutical composition of embodiment 22, wherein the initial loading dose is about 60 mg/kg or about 30 mg/kg, and wherein the maintenance dose is about 15 mg/kg or about 30 mg/kg.
24. The pharmaceutical composition of embodiment 22, wherein the pediatric myasthenia gravis is transient neonatal myasthenia.
25. The pharmaceutical composition of embodiment 22, wherein the pediatric myasthenia gravis is juvenile myasthenia gravis.
26. The pharmaceutical composition of embodiment 22, wherein the pediatric myasthenia gravis is congenital myasthenia gravis syndrome.

The following examples are illustrative, but not limiting, of the methods and compositions described herein. Other suitable modifications and adaptations known to those skilled in the art are within the scope of the following embodiments.

### EXAMPLES

Example 1. Study Design. Phase 2/3, open-label, multicenter study to evaluate the pharmacokinetics (PK), pharmacodynamics (PD), safety, tolerability, efficacy of study drug, and activity of intravenous nipocalimab in pediatric subjects (2 years old to less than 18 years old) with gMG who have an insufficient clinical response to ongoing, stable standard of care therapy.

A screening period of up to 28 days allows for sufficient time to perform screening evaluations and determine study eligibility.

Total of 12 subjects proposed, 6 in each of the two age cohorts: Cohort 1)>=12 years to <18 Years; and Cohort 2)>=2 years to <12 years.

The active treatment phase includes Cohorts 1 and 2, which can be unblinded and open to all male and female pediatric subjects meeting study specific inclusion and exclusion criteria. The study population will be comprised of boys and girls who have had a diagnosis of gMG with generalized muscle weakness, who have an insufficient clinical response to ongoing, stable standard-of-care therapy, and meet the clinical criteria for gMG as defined by the Myasthenia Gravis Foundation of America (MGFA) Clinical Classification Class II a/b, III a/b, or IV a/b at screening. Additionally, participants will have a positive serologic test for a gMG-related pathogenic autoantibody (anti-AChR and/or anti-MuSK).

Pediatric patients will be excluded from the study for the following: 1) Has a history of severe and/or uncontrolled liver, gastrointestinal, renal, pulmonary, cardiovascular, psychiatric, neurological or musculoskeletal disorder or any other medical disorder including laboratory abnormalities, that, in the opinion of the Investigator, might interfere with participant's full participation in the study, or might jeopardize the safety of the participant or the validity of the study results. 2) Has any confirmed or suspected clinical immunodeficiency syndrome not related to treatment of his/her gMG, or has a family history of congenital or hereditary immunodeficiency unless confirmed absent in the participant. 3) Has MGFA Class I disease or presence of MG crisis (MGFA Class V) at screening, history of MG crisis within 1 month of screening, or fixed weakness (and/or ‘burnt out’ MG). 4) Is dependent on gastric tube for nutritional needs or is ventilator-dependent. 5) Is actively undergoing radiation or chemotherapy for an unresected thymoma/malignant thymoma. Participants with stable, benign thymoma (stage I or IIa, for example) for which no treatment has been undertaken in the past 3 years may be allowed following discussion with the sponsor's medical monitor. 6) Has had a thymectomy within 12 months prior to screening, or thymectomy is planned during the Active treatment Phase of the study. 7) Has current or a history of any neurologic disorder other than MG that might interfere with the accuracy of study assessments, including but not limited to any chronic neurodegenerative disease, altered level of consciousness, dementia, abnormal mental status, major congenital neurologic defect, Lambert-Eaton myasthenic syndrome, drug induced MG, or hereditary forms of myasthenic syndrome. 8) Currently has a malignancy or has a history of malignancy within 3 years before screening (with the exception of localized basal cell carcinoma and/or squamous cell carcinoma skin cancer that has been adequately treated with no evidence of recurrence for at least 3 months [defined as a minimum of 12 weeks] before the first study intervention administration or cervical carcinoma in situ that has been treated with no evidence of recurrence for at least 3 months before the first study intervention administration). 9) Has known allergies, hypersensitivity, or intolerance to nipocalimab or its excipients (refer to the IB). 10) Has shown a previous severe immediate hypersensitivity reaction, such as anaphylaxis to therapeutic proteins (e.g., monoclonal antibodies). 11) Has experienced myocardial infarction, unstable ischemic heart disease, or stroke within 12 weeks of screening. 12) Prior/Concomitant Therapy: Is currently taking IgG Fc-related protein therapeutics, or Fc-conjugated therapeutic agents, including factor or enzyme replacement. Has received, rituximab within 6 months prior to first administration of study intervention. Has received a live vaccine within 3 months prior to screening or has a known need to receive a live vaccine during the study, or within at least 3 months after the last administration of study intervention. Has received plasmapheresis, immunoadsorption therapy, or IVIg within 4 weeks prior to baseline. Has another medical condition that requires oral or parenteral corticosteroids unless the dose has been stable for at least 4 weeks prior to baseline and is expected to remain stable during the study. Inhaled, intra-articular, topical or ocular corticosteroids are not exclusionary. Has another medical condition that requires an immunosuppressive agent unless the medication has been used for at least 6 months, the dose has been stable for at least 3 months prior to baseline and the medication and the dose are expected to remain stable during the study. Has previously received nipocalimab. 13) Infections or Predisposition to Infections: Has a severe infection including opportunistic infections (e.g., pneumonia, biliary tract infection, diverticulitis, Clostridium difficile infection, cytomegalovirus, pneumocystosis, aspergillosis, etc.) requiring parenteral anti-infectives and/or hospitalization, and/or is assessed as serious/clinically significant by the Investigator, within 8 weeks prior to Screening. The participant may be re-screened after the 8-week exclusionary period has passed. Has a chronic infection (e.g., bronchiectasis, chronic osteomyelitis, chronic pyelonephritis) or requires chronic treatment with anti-infectives (e.g., antibiotics, antivirals). Tests positive for hepatitis B virus (HBV) infection. Is seropositive for antibodies to hepatitis C virus (HCV), unless they satisfy 1 of the following conditions: Has a history of successful treatment, defined as being negative for HCV RNA at least 24 weeks after completing antiviral treatment, and has a negative HCV RNA test result at screening, OR While seropositive, has a negative HCV RNA test result at least 24 weeks prior to screening and a negative HCV RNA test at screening. History of being human immunodeficiency virus (HIV)1 or HIV2 antibody-positive, or tests positive for HIV at screening.

The study will consist of a screening period of up to 4 weeks, a 24-week Active treatment Phase, and a Long-term Extension (LTE) Phase.

Cohort 1 enrolls adolescents (aged 12 to <18 years) to assess PK, PD, safety and activity of study drug treatment. Twelve weeks after all participants have entered Cohort 1, an interim analysis evaluates PK, PD, and safety data, which, if acceptable, initiates the study in Cohort 2 (participants aged 2 to <12 years). Once adolescents are enrolled, younger subjects aged 2 to <12 years can be enrolled. All subjects are administered study drug via IV infusion over 15-30 minutes every two weeks. Participants in Cohort 1 of the study receive a single 30 mg/kg loading dose followed by 15 mg/kg every 2 weeks [q2w]. Dosing for Cohort 2 is modeled based on all extant PK and PD data available (including adult data from Phase 1 to Phase 3 studies and adolescent data from Cohort 1 of this study) at the time of the interim analysis. A total of at least 12 subjects are studied, with at least 6 in each of the two age cohorts (adolescents and younger children).

Study duration is 24 weeks, with an option to enter a long term extension (LTE) after study completion; subjects who discontinue early or do not enter the LTE after completing the study undergo a safety evaluation 8 weeks after terminating their enrollment. The LTE is expected to be approximately 104 weeks (˜2 years) duration. All Cohort 1 participants in the LTE phase of the study have the option to receive nipocalimab infusion q2w (15 mg/kg) or q4w (30 mg/kg), or change background concomitant medications, based on the Investigator's discretion. Options for changing the Cohort 2 participant's dose and regimen during the LTE phase are modeled and based on all extant PK and PD data available (including adult data from Phase 1 to Phase 3 studies and adolescent data from Cohort 1 of this study) at the time of the interim analysis. No other dosing regimen should be used. Participants who discontinue early or do not enter the LTE after completing the study undergo a Safety Follow-up Visit 8 weeks after their last infusion of study intervention.

To assess the PK and PD of study drug in pediatric participants with gMG blood samples are collected at selected visits in this study. Serum samples for immunogenicity assessment are collected at selected visits in this study. Biomarker samples are collected to evaluate the mechanism of action of study drug or may help to identify population subgroups that respond differently to an intervention.

The duration of the Active treatment Phase (24 weeks) is anticipated to be sufficient for the assessment of activity and safety based on nipocalimab's mechanism of action and results of the adult Phase 2 study demonstrating efficacy on the MG-ADL as early as Week 2 and sustained up to Day 57 (the primary assessment time point in the adult Phase 2 study). The maintenance of nipocalimab's effect, as well as the long-term safety of nipocalimab are further evaluated in the LTE phase.

Primary study parameters/outcome of the study: The effect of nipocalimab on total serum IgG in pediatric participants 2 to <18 years of age with gMG who have an insufficient clinical response to ongoing, stable standard-of-care therapy. The safety and tolerability of treatment with nipocalimab in pediatric participants 2 to <18 years of age with gMG who have an insufficient clinical response to ongoing, stable standard-of-care therapy. The pharmacokinetics of nipocalimab in pediatric participants 2 to <18 years of age with gMG who have an insufficient clinical response to ongoing, stable standard of care therapy. All primary PK and IgG endpoints will be summarized descriptively over time for the evaluable population, and for each age cohort (2 to <12, or 12 to <18 years old).

Secondary Endpoints include: The activity of nipocalimab in gMG as measured by the change from baseline in Myasthenia Gravis—Activities of Daily Living (MG-ADL) efficacy score. The activity of nipocalimab in gMG as measured by the change from baseline in Quantitative Myasthenia Gravis (QMG) efficacy score. The effect on quality of life as measured by the European Quality of Life 5-Dimension Youth (EQ-5D-Y) tool. The effect on fatigue as measured by the Neurological Quality of Life (Neuro-QoL) pediatric fatigue score. All secondary endpoints will be summarized descriptively over time for the evaluable population, and for each age cohort (2 to <12, or 12 to <18 years old).

Exploratory Endpoints include: The relationship between nipocalimab dose, nipocalimab PK, total serum IgG, MG-ADL score and QMG score as assessed by a PK-PD model. The effect of nipocalimab treatment on autoantibody levels (anti-AChR and anti-MuSK). The effect on health-related quality of life as measured by the Pediatric Quality of Life Inventory (PedsQL). All exploratory endpoints will be summarized descriptively over time and for each age cohort (2 to <12, or 12 to <18 years old).

Safety assessments include collection of AEs and SAEs, use of concomitant medications, clinical laboratory testing (including chemistry, hematology, lipid profiles, urinalysis, and testing for total serum IgG and vaccine titers to diphtheria/tetanus), ECGs, vital signs, physical examinations and Tanner staging. Urine pregnancy testing is performed only for girls of childbearing potential. In addition, the emergence of suicidal ideation is assessed using the Columbia-Suicide Severity Rating Scale (C-SSRS). Severe or serious infections, events of hypoalbuminemia (<20 g/L), and opportunistic infections are considered adverse events of special interest (AESI).

Example 2. Nipocalimab is safe and well-tolerated in patient with gMG. A pediatric subject is evaluated, prior to treatment with nipocalimab, utilizing one or more of: physical examination, C-SSRS, vital signs, 12-lead ECG, urinalysis. Additionally, blood and serum is collected for exploratory biomarker analysis, Ig type analysis, and clinical laboratory assessments. A subject with myasthenia gravis or a subject with gMG who have an insufficient clinical response to ongoing, stable standard of care therapy is evaluated utilizing physical examination, C-SSRS, vital signs, 12-lead ECG, urinalysis, blood and serum assessment tests prior to treatment with nipocalimab to generate baseline scores. The subject is administered nipocalimab at a single dose, or a loading dose and a maintenance dose every 2 weeks for 24 weeks. After 24 weeks, and throughout the study, the subject is evaluated for changes in vital signs, clinical laboratory values, and C-SSRS score.

Example 3. Modeling of Intravenous Dosing. Various dosing regimens for M281 are modeled based on clinical data from adolescent patients. The impact of various dosing regimens on Myasthenia Gravis Activities of Daily Living (MG-ADL) is modeled. Among the doses modeled are: about 5 mg/kg, about 10 mg/kg, about 15 mg/kg, about 20 mg/kg, about 25 mg/kg, about 30 mg/kg, about 40 mg/kg, about 45 mg/kg, about 50 mg/kg, about 55 mg/kg, about 60 mg/kg. Based on this modeling, the dosing regimen for the younger patients is derived.

Example 4. Treatment of myasthenia gravis with an anti-FcRn antibody leads to change from baseline on MG-ADL scale. A pediatric subject is evaluated, prior to treatment with nipocalimab, utilizing Myasthenia Gravis—Activities of Daily Living (MG-ADL) scale. A subject with myasthenia gravis or a subject with gMG who have an insufficient clinical response to ongoing, stable standard of care therapy is evaluated utilizing MG-ADL prior to treatment with nipocalimab to generate a MG-ADL score. The subject is administered nipocalimab at a single dose, or a loading dose and a maintenance dose every 2 weeks for 24 weeks. After 24 weeks, and throughout the study at day 1, week 1, week 2, week 3, week 4, week 6, week 8, week 12, week 16, week 18, week 20, week 22, week 23, and week 24, the subject is evaluated for changes in MG-ADL score. After 24 weeks, and throughout the study, the subject is evaluated using MG-ADL scale and found to achieve change from baseline on MG-ADL scale.

Example 5. Treatment of myasthenia gravis with an anti-FcRn antibody leads to change from baseline on QMG and MG-QoL15r scales. A subject is evaluated, prior to treatment with nipocalimab, utilizing QMG and MG-QoL 15r scales. A subject with myasthenia gravis or a subject with gMG who have an insufficient clinical response to ongoing, stable standard of care therapy is evaluated utilizing QMG and MG-QoL 15r prior to treatment with nipocalimab to generate a QMG and MG-QoL 15r score. The subject is administered nipocalimab at a single dose, or a loading dose and a maintenance dose every 2 weeks for 24 weeks. After 24 weeks, and throughout the study at day 1, week 2, week 4, week 8, week 12, week 16, week 20, week 22, and week 24, the subject is evaluated for changes in QMG and MG-QoL 15r scores. After 24 weeks, and throughout the study, the subject is evaluated using QMG and MG-QoL 15r scales and found to achieve change from baseline on QMG and MG-QoL 15r scales.

Example 6. Treatment of myasthenia gravis with an anti-FcRn antibody leads to change from baseline on Neuro-QoL-Pediatric Fatigue, EQ-5D-5Y, MGFA, PGI-C, PGI-S, and PedsQL scales. A pediatric subject is evaluated, prior to treatment with nipocalimab, utilizing Neuro-QoL-Pediatric Fatigue (older cohort only), EQ-5D-5Y (older cohort only), MGFA, PGI-C (older cohort only), PGI-S (older cohort only), and PedsQL scales. A pediatric subject with myasthenia gravis or a subject with gMG who have an insufficient clinical response to ongoing, stable standard of care therapy is evaluated utilizing Neuro-QoL-Fatigue (older cohort only), EQ-5D-5Y (older cohort only), MGFA, PGI-C (older cohort only), PGI-S (older cohort only), and PedsQL scales prior to treatment with nipocalimab to generate a Neuro-QoL-Pediatric Fatigue (older cohort only), EQ-5D-5Y (older cohort only), MGFA, PGI-C (older cohort only), PGI-S (older cohort only), and PedsQL score. The pediatric subject is administered nipocalimab at a single dose, or a loading dose and a maintenance dose every 2 weeks for 24 weeks. After 24 weeks, and throughout the study at day 1 (except for PGI-C), week 2, week 4, week 8, week 12, week 16, week 20, week 22, and week 24, the pediatric subject is evaluated for changes in Neuro-QoL-Pediatric Fatigue, EQ-5D-5Y, PGI-C, PGI-S, and PedsQL scores. After 24 weeks, and throughout the study at day 1 and week 12, the pediatric subject is evaluated for changes in MGFA score. After 24 weeks, and throughout the study, the pediatric subject is evaluated using Neuro-QoL-Pediatric Fatigue, EQ-5D-5Y, MGFA, PGI-C, PGI-S, and PedsQL scales and found to achieve change from baseline on Neuro-QoL-Pediatric Fatigue, EQ-5D-5Y, MGFA, PGI-C, PGI-S, and PedsQL scales.

Example 7. Treatment of pediatric myasthenia gravis with nipocalimab does not elevate cholesterol to clinically significant levels. Elevations in total cholesterol and low-density lipoprotein (LDL) were reported recently with another experimental anti-FcRN antibody that is not nipocalimab in the same pharmacological class of FcRn antagonists. This finding triggered a review of lipid data in Sponsor-completed and ongoing nipocalimab studies. In the Phase 1 healthy volunteer and Phase 2 generalized myasthenia gravis studies, asymptomatic, dose dependent, reversible elevations in nonfasting mean total cholesterol were observed up to 25% of baseline. At the highest dose of 60 mg/kg every 2 weeks (Q2W), the mean percent change in total cholesterol increased to a stable maximum of 21% to 23% above baseline within 1 month of initiation of dosing and declined to near baseline level 1-2 months after the last dose. As a result of these findings, the following assessments are conducted: 1) assessment for lipids (total cholesterol, HDL, calculated LDL, and triglycerides) in fasting and non-fasting conditions at multiple time points on and off treatment, 2) exclusion criterion for patients with a recent significant cardiovascular event, 3) recommendation for lipid abnormalities management according to local health guidelines.

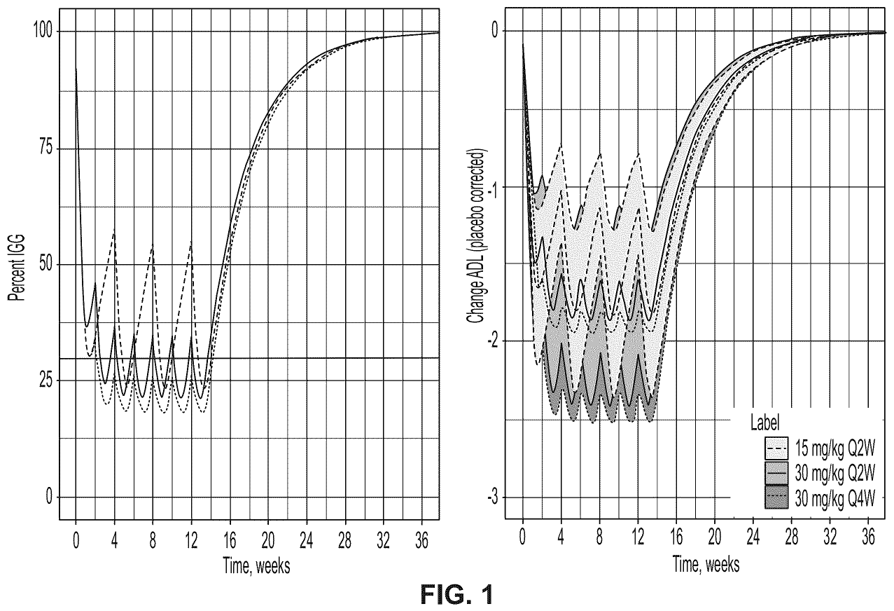

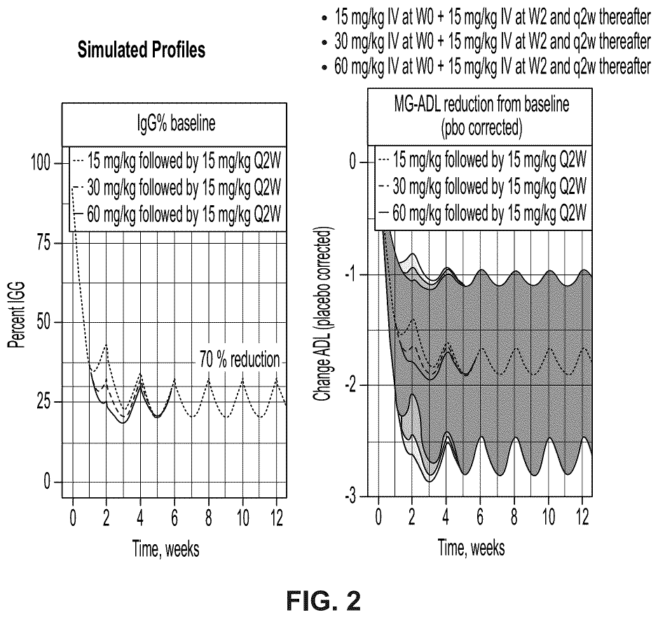

Example 8. Pediatric dose justification. The proposed dose level and dosing regimen for Cohort 1 of this Phase 2/3 study in adolescent participants with gMG (i.e., 30 mg/kg IV loading dose on Day 1 followed by 15 mg/kg IV q2w maintenance doses from Week 2) was based on the dose level and dosing regimen selected for the Phase 3 study in adult participants with gMG. The dose level and dosing regimen for the adult Phase 3 gMG study was based on observed data from the adult Phase 2 study in participants with gMG and extensive modeling and simulation of the dose response relationships for IgG and MG-ADL using data from adult Phase 1 and Phase 2 studies. Dosing for Cohort 2 of the present study, and as proposed in Example 1, is modeled based on all extant PK and PD data available (including adult data from Phase 1 to Phase 3 studies and adolescent data from Cohort 1 of the present study) at the time of the interim analysis. In the adult Phase 2 gMG study, rapid, dose-dependent IgG lowering was observed one week after the initial dose in all dose groups, with maximal IgG lowering achieved at Week 2 in the 60 mg/kg single dose and 60 mg/kg q2w groups. Dose-dependent improvements in MG-ADL scores were also observed, suggesting a correlation between IgG lowering and MG-ADL score improvement. Importantly, nipocalimab was generally well tolerated across all dose groups. Population PK/PD/efficacy modeling analyses were conducted using data obtained from nipocalimab adult Phase 1 and 2 studies to evaluate the relationship between PK, IgG lowering, and MG-ADL, in addition to other efficacy and safety endpoints (including serum albumin and cholesterol). The results indicated that the q2w dosing interval would provide more sustained IgG lowering and MG-ADL reduction at all simulated dose levels when compared with the q4w dosing interval. While modeling and simulation suggested numerical differences in IgG lowering and MG-ADL reduction between the 15 and 30 mg/kg q2w dosing regimens (the model-predicted mean IgG lowering is 73.8% versus 79.4%, respectively), the additional 5.6% IgG reduction with 30 mg/kg q2w translates to minimal additional MG-ADL improvement at steady-state trough beyond the improvement expected with 15 mg/kg q2w (FIG.1). Therefore, the 15 mg/kg q2w dose regimen is selected as the single maintenance dose regimen to be studied for the adult Phase 3 gMG study since this is a rare disease with high unmet need. Lower doses likely result in suboptimal efficacy, while higher doses may not yield much difference in efficacy as predicted for gMG. The predicted exposure with a 30 mg/kg IV loading dose on Day 1 followed by 15 mg/kg IV q2w maintenance doses is well below the PK exposure observed from the 60 mg/kg q2w dosing regimen in the adult Phase 2 gMG study, which was generally well-tolerated based on the currently available safety data. The dosing regimen planned for the adult Phase 3 gMG study is expected to have an average of <20% albumin lowering and <20% total cholesterol increase at steady state. The magnitudes of albumin reduction and total cholesterol increase are not expected to be clinically significant and are less than those observed in prior adult studies with 30 mg/kg IV weekly or 60 mg/kg IV q2w dose regimens. Therefore, the proposed dose for the adult Phase 3 gMG study is expected to be safe and well tolerated. To propose the dose regimen for adolescent participants with gMG, a population PK/receptor occupancy (RO)/IgG model was developed using data from adults and the model was adapted for dose selection in pediatric participants with gMG. Based on data from literature, the adapted model accounted for 1) lower clearance (CL) and volume of distribution (V) in pediatric patients: the CL and V were scaled by body weight according to allometry; and 2) age and/or weight-dependent FcRn and IgG at baseline in pediatric patients: the FcRn and IgG were scaled by age and/or weight (Hardiansyah 2018). The results from the model-based simulation demonstrated comparable PK and IgG profiles between adolescent and adult patients with gMG when treated with the proposed dose and dosing regimen (FIG. 2). Therefore, the dose and dosing regimen selected for the adult Phase 3 gMG study are also used for Cohort 1 of this study in adolescent patients 12 to <18 years of age and no dose adjustment is needed. An interim analysis is performed to evaluate the PK, PD, and safety data after adolescent participants in Cohort 1 complete their first 12 weeks of treatment with nipocalimab. The dose regimen for children (2 to <12 years of age) in Cohort 2 is selected based on PK-PD modeling and simulation using all extant PK and PD data (including adult data from Phase 1 to Phase 3 studies and adolescent data from Cohort 1 of this study) available at the time of interim analysis. In the interim PK-PD modeling and simulation, the CL and V are scaled by body weight or body surface area (BSA) and the FcRn and IgG will be scaled by age, weight, or BSA. Depending on the results from interim analysis, dose adjustment may be needed for children in Cohort 2.

The embodiments and examples provided herein demonstrate that an anti-FcRn antibody, such as, but not limited to, nipocalimab is effective to treat myasthenia gravis as measured one or more of the indices and outcomes as provided for herein.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

What is claimed is:

1. A method of treating pediatric myasthenia gravis in a pediatric patient in need thereof, the method comprising administering an initial loading dose of about 30 mg/kg to about 60 mg/kg of an anti-FcRn antibody followed by administering a maintenance dose of about 15 mg/kg to about 30 mg/kg of the anti-FcRn antibody, wherein the anti-FcRn antibody comprises:
a heavy chain comprising a HCDR1 of SEQ ID NO: 6, a HCDR2 of SEQ ID NO: 7, and a HCDR3 of SEQ ID NO: 8; and
a light chain comprising a LCDR1 of SEQ ID NO: 3, a LCDR2 of SEQ ID NO: 4, and a LCDR3 of SEQ ID NO: 5;
wherein the administration reduces serum IgG in the patient by at least 10%, at least 20%, at least 30%, at least 40%, at least 50%, at least 60%, at least 70%, at least 80%, or at least 90% of baseline serum IgG, and
wherein the pediatric myasthenia gravis is selected from transient neonatal myasthenia, juvenile myasthenia gravis, congenital myasthenia gravis syndrome, or any combination thereof.

2. The method of claim 1, wherein the pediatric myasthenia gravis is transient neonatal myasthenia.

3. The method of claim 1, wherein the pediatric myasthenia gravis is juvenile myasthenia gravis.

4. The method of claim 1, wherein the pediatric myasthenia gravis is congenital myasthenia gravis syndrome.

5. The method of claim 1, wherein the heavy chain comprises an amino acid sequence having at least 95% identity to the sequence of SEQ ID NO: 2 and the light chain comprises an amino acid sequence having at least 95% identity to the sequence of SEQ ID NO: 1.

6. The method of claim 1, wherein the heavy chain comprises a variable region heavy chain comprising an amino acid sequence having at least 95% identity to the sequence of SEQ ID NO: 10 and the light chain comprises a variable region light chain comprising an amino acid sequence having at least 95% identity to the sequence of SEQ ID NO: 9.

7. The method of claim 1, wherein the heavy chain comprises the amino acid sequence of SEQ ID NO: 2 and the light chain comprises the amino acid sequence of SEQ ID NO: 1.

8. The method of claim 1, wherein the heavy chain comprises a variable region heavy chain comprising the amino acid sequence of SEQ ID NO: 10 and the light chain comprises a variable region light chain comprising the amino acid sequence of SEQ ID NO: 9.

9. The method of any one of claims 1-8, wherein the administration is intravenous or subcutaneous.

10. The method of any one of claims 1-9, wherein the administration comprises administering a pharmaceutical composition comprising about 10 mg/ml to about 60 mg/ml of the anti-FcRn antibody, about 20 mM to about 30 mM sodium phosphate, about 20 mM to about 30 mM sodium chloride, about 80 mg/ml to about 100 mg/ml Trehalose, and about 0.1% w/v to about 0.005% w/v Polysorbate 80.

11. The method of any one of claims 1-10, wherein the initial loading dose is about 60 mg/kg or about 30 mg/kg.

12. The method of any one of claim 1-11, wherein the maintenance dose is about 15 mg/kg or about 30 mg/kg.

13. The method of any one of claims 1-12, wherein the maintenance dose is administered:
1 week, 2 weeks, 3 weeks, 4 weeks, or monthly after the administration of the initial loading dose; and
1 week, 2 weeks, 3 weeks, 4 weeks, or monthly after the administration of the preceding maintenance dose.

14. The method of any one of claims 1-13, wherein:
the initial loading dose is infused into the pediatric patient in about 30 minutes to about 90 minutes; and
the maintenance dose is infused into the pediatric patient in about 15 to about 60 minutes.

15. The method of any one of claims 1-14, wherein the serum IgG is IgG1, IgG2, IgG3, or IgG4, or any combination thereof, and wherein the reduction is by at least 20% of baseline, or at least 30% of baseline.

16. The method of any one of claims 1-15, wherein the administration of the anti-FcRn antibody reduces serum albumin by at most 18%, at most 16%, at most 14%, at most 12%, at most 10%, at most 8%, at most 6%, at most 4%, or at most 2% of baseline of serum albumin.

17. The method of any one of claims 1-16, wherein the administration reduces serum autoantibodies, wherein:
the autoantibodies are selected from the group consisting of: anti-acetylcholine receptors (AChRs), anti-muscle-specific kinase (MuSK) anti-low-density lipoprotein receptor-related protein 4 (LRP4), anti-agrin, anti-titin, anti-Kv1.4, anti-ryanodine receptors, anti-collagen Q, and anti-cortactin; and
the reduction is by at least 95%, at least 90%, at least 85%, at least 80%, at least 75%, at least 50%, or at least 25% of baseline serum autoantibodies.

18. The method of claim 17, wherein the administration of the anti-FcRn antibody reduces anti-AChR antibodies by at least 95%, at least 90%, at least 85%, at least 80%, at least 75%, at least 50%, or at least 25% of baseline anti-AChR antibodies.

19. The method of any one of claim 17 or 18, wherein the administration of the anti-FcRn antibody reduces anti-MuSK antibodies by at least 95%, at least 90%, at least 85%, at least 80%, at least 75%, at least 50%, or at least 25% of baseline anti-MuSK antibodies.

20. The method of any one of claims 1-19, wherein the patient achieves a change from baseline in MG-ADL score, QMG score, Neuro-QoL-Fatigue score, EQ-5D-5Y score, PGI-C score, PGI-S score, PedsQL score, or any combination thereof.

21. The method of any one of claims 1-20, wherein the administration of the anti-FcRn antibody to the pediatric patient does not significantly increase levels of total cholesterol, HDL, calculated LDL, and triglycerides in the subject as compared to the levels prior to the administration of the anti-FcRn antibody.

22. A pharmaceutical composition comprising an anti-FcRn antibody for administration to a pediatric patient suffering from pediatric myasthenia gravis, wherein:
the anti-FcRn antibody is administered to the pediatric patient intravenously or subcutaneously at an initial loading dose of about 30 mg/kg to about 60 mg/kg followed by administering a maintenance dose of about 15 mg/kg to about 30 mg/kg of the anti-FcRn antibody; and
the anti-FcRn antibody comprises:
a heavy chain comprising a HCDR1 of SEQ ID NO: 6, a HCDR2 of SEQ ID NO: 7, and a HCDR3 of SEQ ID NO: 8; and
a light chain comprising a LCDR1 of SEQ ID NO: 3, a LCDR2 of SEQ ID NO: 4, and a LCDR3 of SEQ ID NO: 5, and
wherein the pediatric myasthenia gravis is selected from transient neonatal myasthenia, juvenile myasthenia gravis, congenital myasthenia gravis syndromes, or any combination thereof.

23. The pharmaceutical composition of claim 22, wherein the initial loading dose is about 60 mg/kg or about 30 mg/kg, and wherein the maintenance dose is about 15 mg/kg or about 30 mg/kg.

24. The pharmaceutical composition of claim 22, wherein the pediatric myasthenia gravis is transient neonatal myasthenia.

25. The pharmaceutical composition of claim 22, wherein the pediatric myasthenia gravis is juvenile myasthenia gravis.

26. The pharmaceutical composition of claim 22, wherein the pediatric myasthenia gravis is congenital myasthenia gravis syndrome.
